(19) United States
(12) Patent Application Publication
(10) Pub. No.: US 2025/0032468 A1
(43) Pub. Date: Jan. 30, 2025

(54) OCULAR SURFACE TOPICAL ADMINISTRATION OF ROFLUMILAST FOR TREATMENT OF ANTERIOR AND RETINAL-VITREOUS CHAMBER OCULAR DISEASES

(71) Applicant: IOLYX THERAPEUTICS, INC., Burlingame, CA (US)

(72) Inventors: Elizabeth W. JEFFORDS, Hillsborough, CA (US); Shannon L.M. DAHL, Palo Alto, CA (US); Richard GRAHAM, Irvine, CA (US); Hovhannes J. GUKASYAN, Orange, CA (US); Rozemarijn VERHOEVEN, Siler City, NC (US)

(73) Assignee: IOLYX THERAPEUTICS, INC., Burlingame, CA (US)

(21) Appl. No.: 18/783,634

(22) Filed: Jul. 25, 2024

Related U.S. Application Data
(60) Provisional application No. 63/528,674, filed on Jul. 25, 2023.

(51) Int. Cl. A61K 31/44; A61K 9/00; A61K 9/06; A61K 47/10; A61K 47/12; A61K 47/26; A61K 47/32; A61K 47/38

(52) U.S. Cl. CPC A61K 31/44; A61K 9/0048; A61K 9/06; A61K 47/10; A61K 47/12; A61K 47/26; A61K 47/32; A61K 47/38

(57) ABSTRACT

Methods of treating anterior or vitreous chamber ocular diseases by administering ophthalmic pharmaceutical compositions of roflumilast topically to the ocular surface of the eye of a patient. The methods involving topical administration of ophthalmic pharmaceutical compositions of roflumilast to the ocular surface of a patient three or four times a day at more frequent and higher concentration, which can provide significant therapeutic activity to the anterior and vitreous chamber compartments of the eye using dosing regimens involving higher concentrations and less frequent administration than other treatment regimens.

### CROSS REFERENCE TO RELATED APPLICATION

The present application claims priority to U.S. Provisional Application No. 63/528,674, filed Jul. 25, 2023, which is incorporated herein by reference.

### FIELD OF THE INVENTION

The present invention is directed to methods of treating anterior and vitreous chamber (also called the vitreous compartment, posterior chamber, retina-vitreous chamber, or retina or choroid) ocular diseases by administering ophthalmic pharmaceutical compositions of roflumilast topically to the ocular surface of the eye of a patient according to the higher concentration and higher frequency dosing regimens described herein.

### BACKGROUND OF THE INVENTION

Roflumilast is a potent and selective long-acting inhibitor of phosphodiesterase (PDE) type 4, with anti-inflammatory and potential antineoplastic activities. Roflumilast is known to be suitable as a bronchial therapeutic agent as well as for the treatment of inflammatory disorders. Compositions containing roflumilast are used in human and veterinary medicine and have been proposed for the treatment and prophylaxis of diseases including but not limited to: inflammatory and allergen-induced airway disorders (e.g., bronchitis, asthma, COPD), dermatoses (e.g., proliferative, inflammatory, and allergen induced skin disorders), and generalized inflammations in the gastrointestinal region (Crohn's disease and ulcerative colitis). Oral pharmaceutical compositions of roflumilast are currently marketed under the tradenames Daliresp® (in the United States) and Daxas® (in Europe) for COPD, and topical compositions of roflumilast cream for dermatological use are currently marketed under the tradename Zoryve™ (in the United States) for psoriasis and other dermatitic diseases.

Roflumilast and its synthesis are described in U.S. Pat. No. 5,712,298. It has been recognized that pharmaceutical compounds having phosphodiesterase (PDE)-4 inhibiting properties, such as roflumilast, are therapeutically effective and useful for treating inflammatory disorders, such as psoriasis and atopic dermatitis. While the therapeutic effectiveness of oral and dermal pharmaceutical compositions have been studied, there is a need for ophthalmic pharmaceutical compositions of roflumilast suitable for treating anterior ocular diseases.

The delivery of drugs to the eye is very difficult, as pharmaceutical ophthalmic agents must balance tolerability, sterility, safety, and efficacy. Priyanka Agarwal et al., Formulation Considerations for the Management of Dry Eye Disease, Pharmaceutics, 13, 207 (Feb. 3, 2021) discusses the formulation challenges for ophthalmic pharmaceutical formulations. For example, there can be poor tolerability of formulation excipients. Additionally, poor patient compliance is frequently a challenge with ophthalmic pharmaceutical formulations. Developing a stable ophthalmic formulation which can be made under sterile conditions while retaining physico-chemical properties of the active agent, staying within a tight range of pH and inactive ingredients which are tolerable to the eye, and which can be delivered in effective doses to the eye is very difficult. Ophthalmic delivery is focused on either the ocular surface, the anterior chamber, or vitreous chamber of the eye. Formulations delivered topically to the ocular surface, regardless of the site of disease or involved tissue, often delivered by the patient rather than a medical professional, one to four or more times a day, have the additional challenge of requiring dosing consistency and yet flexibility to deliver effective dose despite common operator errors found in home-based patient delivery: sterility issues, variance in delivery volume, and accuracy in placement. Patients with long-term ocular disease also have increased sensitivity to active and inactive ingredients and preservatives, creating additional formulation challenges.

The present application is directed to the treatment of diseases affecting the anterior and vitreous chambers, including retinal and choroid ocular diseases. Anterior ocular diseases are those affecting the anterior compartment of the eye, including the corneal endothelium, the aqueous humor, the lens, pupil, iris, ciliary body, anterior portions of the lacrimal gland and lymph nodes, and anterior sclera. Anterior ocular diseases are in contrast to vitreoretinal, also referred to as vitreous compartment ocular diseases, such as those affecting the posterior sclera, retina, choroid, macula, fovea, optic disc, optic nerve, vitreous humor, and hyaloid canal; or the ocular surface diseases which impact the corneal epithelium or stroma, limbus, conjunctiva, eyelid, Meibomian glands, and other tissues exposed to the exterior of the body. The anterior chamber is particularly difficult to reach with pharmaceutical treatment due to the inability or inefficiency with which many common pharmaceutical agents transit across alternating hydrophobic and hydrophilic layers of the cornea, despite its attractiveness as the most direct anatomic and geometric route of penetration to the anterior chamber. Although there is some fluid transit from anterior to posterior to vitreous chamber, the lens itself is impermeable, and pharmaceutical delivery to the vitreous compartment is typically achieved via injection or implant directly to vitreoretinal tissues. Furthermore, the bathing of the anterior chamber by the aqueous humor is subject to frequent turnover and fluid drainage unless limited by glaucoma or conditions limiting fluid transit, creating the need for strong pharmacokinetics and maintenance of a high therapeutic level of drug for sustained effect. Despite the difficulty of delivering pharmaceutics to the anterior or vitreous chambers, they are immune-rich environments, as the ICB, sclera, retina and choroid are rich in both vascular and lymphatic access, enabling frequent t-cell and other inflammatory cell-trafficking. As such, the anterior and vitreous chambers are both frequent sources of inflammation in the eye, and yet difficult to reach and treat with conventional topical therapeutics. An exemplary anterior ocular disease found in both the anterior and vitreous chamber subject to such inflammation is uveitis. Uveitis is a form of eye inflammation that can cause permanent vision loss, and is frequently recurrent. It is further classified by the site of the inflammation: anterior, posterior, or when involving multiple locations: pan or intermediate.

The literature suggests a correlation between certain cytokines (e.g., Th1, Th2, and Th17) and certain anterior and intraocular disorders. For example, Th1 and Th2 cytokine responses are suggested to be involved with ocular cicatrical pemphigold (OCP). (Stern 2020, FIG. 3.) The overabundance of anti-inflammatory factors, namely transforming growth factor (TGF)-β, produced by ocular surface epithelial cells and innate effectors also contributes directly to the pathology of OCP. (Stern 2020.) Interleukin (IL)-13 has also been shown to be an important contributor to conjunctival fibrosis and inflammation in OCP. (Stern 2020.) Together these factors can cause tissue scarring, which ultimately can lead to blindness. (Stern 2020.)

As another example, uveitis is often associated with systemic autoimmune diseases and genetic factors such as human leukocyte antigen B27 (HLA-B27), both of which are correlated to Th1 and Th17 cytokine responses, which have been reported to be increased in uveitis patients. For example, Ooi 2006 reported that key Th1 (e.g., IFNg, TNFa) and Th17 (e.g., IL-6) cytokines are detected in patients with uveitis. (Ooi 2006). Additionally, key Th1 (e.g., IFNg, TNFa) and Th17 (e.g., IL-17, IL-6) cytokines have been detected in patients with non-infectious uveitis, including the serum, aqueous humor, and/or vitreous of patients. (Balamurugan 2020). Autoimmune and many idiopathic non-infectious uveitis patients display activated Th1 and Th17 cells, increased associated cytokines, and activated macrophages (associated with Th1 response) and neutrophils (associated with Th17 response). (Takeuchi 2021, Ozgonul 2016, Chen 2015, Errera 2022, Balamurugan 2020). Disease severity in acute anterior uveitis (AAU) with human leukocyte antigen B27 (HLA-B27) also correlates with: (1) increased Th17 cells; (2) decreased Treg; and (3) increased Th17/Treg ratio. (Zhuang 2017).

Existing standard of care therapies for anterior or intraocular diseases often include treatment with corticosteroids. Corticosteroids, although predominantly Th1 targeted (Schewitz-Bowers 2015), are relatively effective with a short onset of action, can be administered in a multitude of formulations: topical suspensions, gels, ointments, injections, implants or depots; and can be administered in co-formulations with antibiotics or other targeted pharmaceutical agents. Corticosteroids administered to the ocular surface permeate into the anterior chamber by diffusion across the cornea into the aqueous humor, which causes them to be less durable and subject to shorter tissue residence (Fung 2020) resulting in the need for more frequent dosing.

And yet, much like with their use in dermatological, immunological, autoimmune, and a host of other disease states; corticosteroids, even in short-term settings, come with a variety of profound local and systemic side effects. Topical corticosteroids for ocular use are label-restricted for short-term use only and can result in eye itching, aches and pains, and other side effects commonly associated with corticosteroids. Systemic corticosteroids can have an impact on increasing intraocular pressure, which can lead to glaucoma, optic nerve damage, cataracts or central serous chorioretinopathy (Fung 2020). Instillation related effects can include site related pain, burning or stinging, allergic reactions, foreign body sensation, visual disturbance, pruritis, urticaria, and rash; as well as keratitis, conjunctivitis, corneal ulcers, mydriasis, hyperemia, loss of accommodation, ptosis, acute anterior uveitis and perforation of the globe. (Pred Forte Label, 2017). Systemic side effects can include headache, elevated blood glucose and susceptibility to systemic microbial infections. Ocular injections and corticosteroid implants with a longer durability have a greater risk of cataracts, increased intraocular pressure, and glaucoma in a significant share of patients.

These side effects can have a direct impact to patient quality of life (pain, discomfort, itching), which are exacerbated by the fact that these products are often given four to eight times a day or even more often, particularly at the beginning of their use, and often need to be tapered to lower doses because of these side effects. In clinical practice, it is not uncommon to use a corticosteroid every 1 to 2 hours for several weeks, then tapering down to labeled doses, for indications like anterior uveitis, to attempt to achieve an adequate clinical effect on the underlying inflammation inherent in this immune-mediated disease. One reason for this extreme frequency of use is to achieve the desired pharmacokinetic distribution and therapeutic levels in the anterior chamber compartment. For pan or posterior uveitis or other intraocular inflammatory diseases, corticosteroid injections or implants are more commonly used, as topical steroids typically do not reach the relevant vitreoretinal tissues in required therapeutic doses.

### SUMMARY OF THE INVENTION

The present application addresses the unmet need for a safe and effective topical therapy for anterior and vitreous chamber intraocular diseases. Ideal therapies can provide adequate pharmacokinetic coverage of disease targets while providing reduced side effects and improved patient preference and compliance. Thus, the present invention provides for an effective alternative to existing therapies, such as those involving corticosteroids.

The present application also provides for methods that include administration of optimal concentrations of roflumilast that are administered at frequencies capable of achieving drug levels sufficiently above IC50 levels in the anterior and vitreous chamber. The concentrations of roflumilast capable of achieving drug levels sufficiently above IC50 levels in the anterior and vitreous chamber are higher than the roflumilast concentrations necessary to obtain similar levels in ocular surface tissues (e.g. the cornea and/or conjunctiva) and are administered on a more frequent basis, and yet at a lower concentration and frequency than other ophthalmic agents for the anterior and vitreous chambers such as corticosteroids.

The present invention includes methods of treating anterior and vitreous chamber ocular diseases by administering ophthalmic pharmaceutical compositions of roflumilast topically to the ocular surface of the eye of a patient in a concentration and frequency optimized for therapeutic coverage of PDE4A, B, and D with the goal of downregulating Th17 and Th1 immune targets in the anterior chamber compartment as well as the vitreous chamber. The inventors of the subject application have surprisingly discovered that topical administration of higher concentration, higher frequency ophthalmic pharmaceutical compositions of roflumilast applied to the ocular surface can provide more significant therapeutic activity and coverage to the tissues of the anterior and vitreous chamber of the eye than those concentrations and frequencies of topical ophthalmic pharmaceutical compositions of roflumilast used for the ocular surface, but which nonetheless still result in less frequent administration than other treatment regimens currently used, such as steroids. The present invention addresses the long felt and unmet need for an alternative class of agents to address anterior ophthalmic disorders using a dosing regimen involving a patient-convenient three or four times a day administration of high concentrations of roflumilast with clinically meaningful efficacy and with lower patient-based or population-based side effects. With high levels of efficacy and minimal side effects, the present invention can provide a short-term, mid-term, or long-term therapeutic solution for anterior ocular diseases.

In certain embodiments, a method for treating a patient having an anterior ophthalmic disorder is provided. The method comprises topically administering an ophthalmic pharmaceutical composition comprising 0.3% to 1.0% w/v of roflumilast or a pharmaceutically acceptable salt thereof to an ocular surface of said patient three times per day. The anterior ophthalmic disorder can be selected from the group consisting of anterior uveitis, pan-uveitis, intermediate uveitis, uveitis associated with various autoimmune or systemic diseases including juvenile idiopathic arthritis (JIA), Behçet's disease, ankylosing spondylitis, sarcoidosis, Vogt-Koyanagi-Harada disease (VKH), lupus, rheumatoid arthritis (RA), multiple sclerosis or other autoimmune conditions; Idiopathic uveitis, HLA-B27 uveitis, uveitic glaucoma, iritis, iridocyclitis, rubeosis iritis, coloboma of the iris, Fuchs heterchromic iridocyclitis, iridocorneal endothelial syndrome (ICE), scleritis, corneal endothelial disease or dysfunction including endotheliitis, Fuch's corneal or endothelial dystrophy, endothelial corneal dysfunction associated with Graves ophthalmopathy or thyroid eye disease, megalocornea, Axenfeld's syndrome, Rieger's anomaly, Peters' anomaly or Anterior segment mesenchymal dysgenesis, ocular hypertension, glaucoma (including primary congenital, open angle, angle closure, normal tension, elevated episcleral venous pressure (EVP), Sturge Weber, or secondary glaucoma), bupthalmos, Posner Schlossman Syndrome or glaucomatocyclitic crisis, cataract, post-surgical anterior ocular inflammation, post-surgical ocular inflammation affecting corneal endothelium, deep corneal epithelial defects that also involve anterior segment dysfunction such as corneal endothelial dysfunction or neurotrophic keratitis, inflammation related to DSEK/DMET keratoplasty, rebound iritis, pseudoexfoliation syndrome, pupillary abnormalities, Stevens Johnson syndrome, Sjogren's syndrome, ocular graft-versus-host disease, ocular cicatricial pemphigoid, anterior sterile or infectious endophthalmitis, toxic anterior segment syndrome (TASS), sympathetic ophthalmia, hyphema, anterior segment ischemia, anterior chamber inflammation related to the underlying viral and other microbial causes, anterior chemical injury, and iatrogenic anterior chamber inflammation. In preferred embodiments, the anterior chamber ophthalmic disorder is uveitis (anterior, pan, intermediate or posterior) or uveitis associated with HLA-B27, autoimmune or systemic disease (including but not limited to JIA, Behçet's, ankylosing spondylitis, VKH lupus, RA, and psoriatic arthritis), uveitic glaucoma, anterior chamber iatrogenic inflammation, ocular pemphigoid, or intraocular inflammatory disease of the corneal endothelium, iris, sclera, or choroid. The method can improve patient convenience and/or result in a reduction of at least one side effect relative to administration of a corticosteroid.

In certain embodiments, a method for treating a patient having an anterior ophthalmic disorder is provided. The method comprises topically administering an ophthalmic pharmaceutical composition comprising 0.3% to 1.0% w/v of roflumilast or a pharmaceutically acceptable salt thereof to an ocular surface of said patient four times per day. The anterior ophthalmic disorder can be selected from the group consisting of anterior uveitis, pan-uveitis, intermediate uveitis, uveitis associated with various autoimmune or systemic diseases including juvenile idiopathic arthritis (JIA), Behçet's disease, ankylosing spondylitis, sarcoidosis, Vogt-Koyanagi-Harada disease (VKH), lupus, rheumatoid arthritis (RA), multiple sclerosis or other autoimmune conditions, idiopathic uveitis, HLA-B27 uveitis, uveitic glaucoma, iritis, iridocyclitis, rubeosis iritis, coloboma of the iris, Fuchs heterchromic iridocyclitis, iridocorneal endothelial syndrome (ICE), scleritis, corneal endothelial disease or dysfunction including endotheliitis, Fuch's corneal or endothelial dystrophy, endothelial corneal dysfunction associated with Graves ophthalmopathy or thyroid eye disease, megalocornea, Axenfeld's syndrome, Rieger's anomaly, Peters' anomaly or Anterior segment mesenchymal dysgenesis, ocular hypertension, glaucoma (including primary congenital, open angle, angle closure, normal tension, elevated episcleral venous pressure (EVP), Sturge Weber, or secondary glaucoma, bupthalmos, Posner Schlossman Syndrome or glaucomatocyclitic crisis, cataract, post-surgical ocular inflammation, post-surgical anterior ocular inflammation affecting corneal endothelium, deep corneal epithelial defects that also involve anterior segment dysfunction such as corneal endothelial dysfunction or neurotrophic keratitis, inflammation related to DSEK/DMET keratoplasty, rebound iritis, pseudoexfoliation syndrome, pupillary abnormalities, Stevens Johnson syndrome, Sjogren's syndrome, ocular graft-versus-host disease, ocular cicatricial pemphigoid, anterior sterile or infectious endophthalmitis, toxic anterior segment syndrome (TASS), sympathetic ophthalmia, hyphema, anterior segment ischemia, anterior chamber inflammation related to the underlying viral and other microbial causes, anterior chemical injury, and iatrogenic anterior chamber inflammation.

In preferred embodiments, the anterior chamber ophthalmic disorder is uveitis (anterior, pan, intermediate or posterior) or uveitis associated with HLA-B27, autoimmune or systemic disease (including but not limited to JIA, Behçet's, ankylosing spondylitis, VKH lupus, RA, and psoriatic arthritis), uveitic glaucoma, anterior chamber iatrogenic inflammation, ocular pemphigoid, or intraocular inflammatory disease of the corneal endothelium, iris, sclera, or choroid. The method can improve patient convenience and/or result in a reduction of at least one side effect relative to administration of a corticosteroid.

In certain embodiments, a method for treating a patient having a vitreous ophthalmic disorder is provided. The method comprises topically administering an ophthalmic pharmaceutical composition comprising 0.3% to 1.0% w/v of roflumilast or a pharmaceutically acceptable salt thereof to an ocular surface of said patient three times per day. The vitreous ophthalmic disorder can be selected from the group consisting of posterior, pan or intermediate uveitis or uveitis associated with HLA-B27, juvenile idiopathic arthritis, Behcets disease, ankylosing spondylitis, VKH, or other autoimmune disease, diabetic retinopathy, diabetic macular edema, retinal vein occlusion, age-related macular degeneration including dry, geographic atrophy, or exudative AMD, choroidal neovascularization, choroidal thickening associated with thyroid eye disease, Coat's disease, central serous retinopathy or chorioretinopathy, sterile or infectious endopthalmitis, inflammation associated with inherited retinal diseases, retinitis pigmentosa, Stargardt disease, Leber congenital amaurosis, Leber hereditary optic neuropathy, Usher syndrome, X-linked retinoschisis, choroidemia, zonal occult outer retinopathy, myopia, vitreomacular adhesion, retinal detachment, choroidal detachment and hemorrhage, choroidal rupture, choroidal folds, proliferative vitreoretinopathy, idiopathic ischemia, achromatopsia, retinopathy of prematurity, gyrate atrophy, central areolar choroidal dystrophy, punctate inner choroidopathy, multifocal choroiditis, choroiditis, choroidal granuloma, choroidal dystrophy, choroidal fibrosis, acute posterior multifocal placoid pigment epitheliopathy, serpiginous choroidopathy, birdshot retinochoroidopathy, multiple evanescent white dot syndrome, retinoblastoma, choroidal melanoma, retinal lymphoma, and iatrogenic posterior or vitreous chamber inflammation.

In preferred embodiments, the vitreous chamber ophthalmic disorder is posterior, pan or intermediate uveitis or uveitis associated with HLA-B27, juvenile idiopathic arthritis, Behcets disease, ankylosing spondylitis, VKH, or other autoimmune disease, diabetic retinopathy, diabetic macular edema, age-related macular degeneration including dry, geographic atrophy, or exudative AMD and choroidal neovascularization, choroidal thickening associated with thyroid eye disease, sterile or infectious endophthalmitis, and vitreous or posterior chamber iatrogenic inflammation. The method can improve patient convenience and/or result in a reduction of at least one side effect relative to administration of a corticosteroid.

In certain embodiments, a method for treating a patient having a vitreous ophthalmic disorder is provided. The method comprises topically administering an ophthalmic pharmaceutical composition comprising 0.3% to 1.0% w/v of roflumilast or a pharmaceutically acceptable salt thereof to an ocular surface of said patient four times per day. The vitreous ophthalmic disorder can be selected from the group consisting of posterior, pan or intermediate uveitis or uveitis associated with HLA-B27, Juvenile Idiopathic Arthritis, Behcets disease, ankylosing spondylitis, VKH, or other autoimmune disease, diabetic retinopathy, diabetic macular edema, retinal vein occlusion, age-related macular degeneration including dry, geographic atrophy, or exudative AMD, choroidal neovascularization, choroidal thickening associated with thyroid eye disease, Coat's disease, central serous retinopathy or chorioretinopathy, sterile or infectious endopthalmitis, inflammation associated with inherited retinal diseases, retinitis pigmentosa, Stargardt disease, Leber congenital amaurosis, Leber hereditary optic neuropathy, Usher syndrome, or X-linked retinoschisis, choroidemia, zonal occult outer retinopathy, myopia, vitreomacular adhesion, retinal detachment, choroidal detachment and hemorrhage, choroidal rupture, choroidal folds, proliferative vitreoretinopathy, idiopathic ischemia, achromatopsia, retinopathy of prematurity, gyrate atrophy, central areolar choroidal dystrophy, punctate inner choroidopathy, multifocal choroiditis, choroiditis, choroidal granuloma, choroidal dystrophy, choroidal fibrosis, acute posterior multifocal placoid pigment epitheliopathy, serpiginous choroidopathy, birdshot retinochoroidopathy, multiple evanescent white dot syndrome, retinoblastoma, choroidal melanoma, retinal lymphoma, and iatrogenic posterior or vitreous chamber inflammation.

In preferred embodiments, the vitreous chamber ophthalmic disorder is posterior, pan or intermediate uveitis or uveitis associated with HLA-B27, Juvenile Idiopathic Arthritis, Behcets disease, ankylosing spondylitis, VKH, or other autoimmune disease, diabetic retinopathy, diabetic macular edema, age-related macular degeneration including dry, geographic atrophy, or exudative AMD and choroidal neovascularization, choroidal thickening associated with thyroid eye disease, sterile or infectious endophthalmitis, and vitreous or posterior chamber iatrogenic inflammation. The method can improve patient convenience and/or result in a reduction of at least one side effect relative to administration of a corticosteroid.

### BRIEF DESCRIPTION OF THE DRAWINGS

The accompanying drawings, which are incorporated herein and form part of the disclosure, help illustrate various embodiments of the present invention and, together with the description, further serve to describe the invention to enable a person skilled in the pertinent art to make and use the embodiments disclosed herein. The error bars in the drawings are standard deviation values.

FIG. 1 provides data from two studies comparing the concentration of roflumilast (ng/g) in the iris ciliary body as a function of time following: (a) a 1% w/v roflumilast formulation administered three times a day (TID); (b) a 0.3% w/v roflumilast formulation administered three times a day (TID); or (c) a 0.1% w/v roflumilast formulation administered two times a day (BID).

FIG. 2 provides data from two studies comparing the concentration of roflumilast (ng/mL) in the aqueous humor as a function of time following: (a) a 1% w/v roflumilast formulation administered three times a day (TID); (b) a 0.3% w/v roflumilast formulation administered three times a day (TID); or (c) a 0.1% w/v roflumilast formulation administered two times a day (BID).

FIG. 3 provides data from two studies comparing the concentration of roflumilast (ng/g) in the retina/choroid/retina pigment epithelium (RPE) as a function of time following: (a) a 1% w/v roflumilast formulation administered three times a day (TID); (b) a 0.3% w/v roflumilast formulation administered three times a day (TID); or (c) a 0.1% w/v roflumilast formulation administered two times a day (BID).

FIG. 4 provides a predictive model showing the change in fraction of roflumilast (% FA) absorbed in the cornea and conjunctiva defined as AUC 0-0.5 hrs as a function of the concentration of roflumilast (% w/v), in two predictive models: one using only corneal exposure at the 0.1% dose, and an optimized model including actual exposure data from multiple tissues (anterior, and vitreous chambers) from TID dosing of 0.3 or 1% roflumilast.

FIG. 5 provides data illustrating the actual concentration of roflumilast (ng/g) in the cornea in an actual experiment of a terminal dose following steady state 0.1% roflumilast BID PK for four days plus a single dose on day 5, versus a predictive modeled extrapolation of expected roflumilast concentration when given BID.

FIG. 6 provides a predictive model showing the area under the curve (AUC) from 0-last (ng/g) following administration of roflumilast as a function of the concentration of roflumilast (% w/v) in the anterior chamber tissues AH and ICB in two predictive models: one using only cornea exposure at the 0.1% BID dose, and the optimized model using actual exposure data from TID dosing on days 1-4 followed by QD dosing on day 5 of 0.3 or 1% in multiple tissues and compartments.

FIG. 7 provides a predictive model showing the area under the curve (AUC) from 0-last (ng/g) following administration of roflumilast as a function of the concentration of roflumilast (% w/v) in the vitreous chamber tissues retina/choroid in two predictive models: one using only cornea exposure of the 0.1% BID dose, and an optimized model using actual exposure data from TID dosing of 0.3 or 1% in multiple tissues and compartments.

FIG. 8 provides a comparison of data from FIGS. 1 and 2 illustrating the actual concentration of roflumilast (ng/g) in the anterior compartment tissues iris ciliary body (ICB) and aqueous humor (AH) by increased concentration and frequency, compared with simulated levels in the same tissues, as predicted by the original pharmacokinetic model using only the 0.1% BID PK data from the cornea and common topical ophthalmic data sets.

FIG. 9 provides a comparison of data from FIG. 3 illustrating the concentration of roflumilast (ng/g) in the vitreous compartment tissue of retina/choroid/retinal pigment epithelium (RPE) tissue by increased concentration and frequency, compared with simulated levels in the same tissues, as predicted by a pharmacokinetic model using the original 0.1% BID PK data from the cornea and common topical ophthalmic data sets.

FIG. 10 provides a graph with an optimized simulated exposure level showing therapeutic coverage of the IC50 of even the low dose roflumilast 0.1% in the iris ciliary body (ICB).

FIG. 11 provides data for the clinical response of body weight following three times a day (TID) administration of: (1) vehicle placebo; (2) a 0.1% w/v roflumilast suspension; (3) a 0.3% w/v roflumilast suspension; and (4) a 1.0% w/v roflumilast suspension, as measured in a 13-week dog (beagle) non-clinical model.

FIG. 12 provides data for the clinical response of intraocular pressure following three times a day (TID) administration of: (1) vehicle placebo; (2) a 0.1% w/v roflumilast suspension; (3) a 0.3% w/v roflumilast suspension; and (4) a 1.0% w/v roflumilast suspension, as measured in a 13-week dog (beagle) non-clinical model.

FIG. 13 provides data for the clinical response of central corneal thickness following three times a day (TID) administration of: (1) vehicle placebo; (2) a 0.1% w/v roflumilast suspension; (3) a 0.3% w/v roflumilast suspension; and (4) a 1.0% w/v roflumilast suspension, as measured in a 13-week dog (beagle) non-clinical model.

FIG. 14 provides data for the clinical response of body weight following three times a day (TID) administration of: (1) vehicle placebo; (2) a 0.1% w/v roflumilast suspension; (3) a 0.3% w/v roflumilast suspension; and (4) a 1.0% w/v roflumilast suspension, as measured in a 13-week rabbit (New Zealand white) non-clinical model.

FIG. 15 provides data for the clinical response of intraocular pressure following three times a day (TID) administration of: (1) vehicle placebo/saline; (2) a 0.1% w/v roflumilast suspension; (3) a 0.3% w/v roflumilast suspension; and (4) a 1.0% w/v roflumilast suspension, as measured in a 13-week rabbit (New Zealand white) non-clinical model.

FIG. 16 provides data for the clinical response of central corneal thickness following three times a day (TID) administration of: (1) vehicle placebo/saline; (2) a 0.1% w/v roflumilast suspension; (3) a 0.3% w/v roflumilast suspension; and (4) a 1.0% w/v roflumilast suspension, as measured in a 13-week rabbit (New Zealand white) non-clinical model.

### DETAILED DESCRIPTION OF THE INVENTION

It is to be understood that the invention is not limited to the particular methodology, protocols, and reagents described herein as these may vary. It is also to be understood that the terminology used herein is for the purpose of describing particular embodiments only and is not intended to limit the scope of the present invention which will be limited only by the appended claims. Unless defined otherwise, all technical and scientific terms used herein have the same meanings as commonly understood by one of ordinary skill in the art to which this invention belongs.

All publications, patents and patent applications cited herein are hereby incorporated by reference in their entirety unless otherwise stated. Where the same term is defined in a publication, patent, or patent application and the present disclosure incorporated herein by reference, the definition in the present disclosure represents a controlling definition. For publications, patents and patent applications referenced to describe a particular type of compound, chemistry, etc., the portion relating to such compounds, chemistry, etc. is the portion of the literature incorporated herein by reference.

Note that as used herein, the singular forms “a,” “an,” and “the” include plural referents unless the context clearly dictates otherwise. Thus, for example, “active ingredient” includes a single ingredient and two or more different ingredients.

The term “about” when used in connection with a numerical value is meant to encompass numerical values within a range having a lower limit that is 5% smaller than the indicated numerical value and having an upper limit that is 5% larger than the indicated numerical value.

The term “anterior chamber” or “anterior chamber ocular disease” refers to the intraocular tissues relevant to, and disease affecting the anterior or front of the eye through the iris ciliary body and lens. Anterior ocular diseases are in contrast to vitreous chamber ocular diseases, such as those affecting the retina, and distinct from the ocular surface or those directly impacting external environment facing tissues such as the corneal epithelium or conjunctiva. The term “vitreous chamber” or “vitreous chamber ocular disease” refers to the tissues relevant to, or disease affecting the vitreous chamber located from the posterior of the lens, through and including retina and choroid, and to the optic nerve.

The term “effective” refers to an amount of a compound, agent, substance, formulation or composition that is of sufficient quantity to result in a decrease in severity of disease symptoms, an increase in frequency and duration of disease symptom-free periods, or a prevention of impairment or disability due to the disease affliction. The amount may be as a single dose or according to a multiple dose regimen, alone or in combination with other compounds, agents or substances. One of ordinary skill in the art would be able to determine such amounts based on such factors as a subject's size, the severity of a subject's symptoms, and the particular composition or route of administration selected.

The term “eye disorder,” “eye condition,” or “ocular disorder,” or “intraocular disorder” refer to diseases/conditions of the eye(s) that can be sight-threatening, lead to eye discomfort, and may signal systemic health problems. The ocular surface is composed of the cornea (including particularly the corneal epithelium and stroma), limbus, conjunctiva, eyelids, lacrimal and Meibomian glands, and the interconnecting surface nerves. The anterior chamber is made up of the lens, pupil, iris, anterior sclera, ciliary body, lacrimal glands, aqueous humor, and the internal or endothelial or inner layer of the cornea, lacrimal glands and lymph nodes. The vitreous chamber is made up of the posterior sclera, retina, choroid, macula, fovea, optic disc, optic nerve, vitreous humor, and hyaloid canal. The eye as a whole is supported by the various in-eye and extra-orbital muscles and ligaments.

“Pharmaceutically acceptable” means generally safe for administration to humans or animals. Preferably, a pharmaceutically acceptable component is one that has been approved by a regulatory agency of the Federal or a state government or listed in the U.S. Pharmacopeia, published by the United States Pharmacopeial Convention, Inc., Rockville Md., or other generally recognized pharmacopeia for use in animals, and more particularly in humans.

A “pharmaceutical composition” according to the invention may be present in the form of a composition, wherein the different active ingredients and diluents and/or carriers are admixed with each other, or may take the form of a combined preparation, where the active ingredients are present in partially or totally distinct form. An example for such a combination or combined preparation is a kit-of-parts.

The term “roflumilast” as used in this application refers to roflumilast, its salts, the N-oxide of roflumilast, and its salts and other hydrolytic or amide metabolites unless specified otherwise or unless it is clear in context that reference is to roflumilast itself.

As used herein, the terms “subject” or “patient” most preferably refers to a human being. The terms “subject” or “patient” may include any mammal that may benefit from the compounds described herein.

A “therapeutic amount” or “therapeutically effective amount” is an amount of a therapeutic agent sufficient to achieve the intended purpose. The effective amount of a given therapeutic agent will vary with factors such as the nature of the agent, the route of administration, the size of the subject to receive the therapeutic agent, and the purpose of the administration. The effective amount in each individual case may be determined empirically by a skilled artisan according to established methods in the art.

As used herein, “treat,” “treating,” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

The present invention includes methods of treating anterior and vitreous chamber ocular diseases by administering ophthalmic pharmaceutical compositions of roflumilast topically to the ocular surface of the eye of a patient in a concentration and frequency optimized for therapeutic coverage of PDE4a, b, and d with the goal of downregulating Th17 and Th1 immune targets in the anterior chamber or compartment and vitreous chamber or compartment. The inventors of the subject application have surprisingly discovered that topical administration of ophthalmic pharmaceutical compositions of roflumilast to the ocular surface can provide significant therapeutic coverage and activity to the anterior and vitreous chambers using dosing regimens involving higher concentrations and frequencies of roflumilast than those used for ocular surface disorders, while still at less frequent administration than other current treatment regimens. The present invention addresses the long felt and unmet need for an alternative class of agents to address anterior and vitreous chamber ophthalmic disorders using a dosing regimen involving three or four times a day administration of high concentrations of roflumilast with clinically meaningful efficacy and with lower patient-based or population-based side effects. The present invention can provide a short-term, mid-term, or long-term therapeutic solution for anterior and vitreous ocular diseases with a low level of side effects.

Roflumilast is a compound of the formula (I):

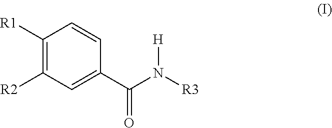

wherein R¹ is difluoromethoxy, R2 is cyclopropylmethoxy and R3 is 3,5-dichloropyrid-4-yl.

Roflumilast has the chemical name N-(3,5-dichloropyrid-4-yl)-3-cyclopropylmethoxy-4-difluoromethoxybenzamide. The N-oxide of roflumilast has the chemical name 3-cyclopropylmethoxy-4-difluoromethoxy-N-(3,5-dichloropyrid-4-yl 1-oxide)benzamide. Roflumilast and its synthesis, the use of roflumilast as a phosphodiesterase (PDE) 4 inhibitor, and roflumilast formulations, were described in U.S. Pat. No. 5,712,298, which is incorporated herein by reference. The ophthalmic pharmaceutical composition can include roflumilast as a free base or a pharmaceutically acceptable salt. Exemplary salts of roflumilast are salt described in paragraphs and of U.S. Patent Application Publication No. US 2006/0084684, the disclosure of which is incorporated herein by reference. In certain embodiments, the pharmaceutical composition comprises a metabolite of roflumilast, including the N-oxide of the pyridine residue of roflumilast or salts thereof, as an active ingredient. In certain embodiments, the pharmaceutical composition comprises a hydrolytic or amide metabolite of roflumilast.

In certain embodiments, the method comprises administering, to a patient suffering from an anterior or vitreous chamber ocular disease, an ophthalmic pharmaceutical composition comprising a therapeutically effective amount of the phosphodiesterase-4 inhibitor, roflumilast or a pharmaceutically acceptable salt or metabolite thereof. In certain embodiments, the ophthalmic pharmaceutical composition is administered three or four times a day (TID or QID).

The present inventors have discovered the methods disclosed herein result in increased concentration in the uveal and anterior tissues (e.g., the iris, ciliary bodies, anterior sclera, aqueous humor, lens, pupil, corneal endothelium, etc.) with a topical formulation capable of achieving lateral migration of roflumilast through the eye and related pharmacokinetics. The methods are able to provide a higher and more consistent level of roflumilast to the anterior chamber tissues with long-lasting pharmacokinetics in a safer and more convenient manner. Additionally, the methods described herein are well-tolerated in contrast to existing standard therapies such as corticosteroids. The methods can provide for high concentrations of roflumilast to be dosed without corresponding safety or side-effect issues.

Unlike the pharmacokinetic properties of steroids for use in the anterior and vitreous compartments, which have a shorter half-life and require high frequency dosing to provide adequate therapeutic coverage of inflammatory targets, the present invention requires only once to four times a day application. A common treatment for relevant ocular inflammation in anterior compartments, Loteprednol 0.5% and Loteprednol-MPP (in a microparticle vehicle) 0.4%, provide steroidal tissue residence which rapidly decreases to the IC50 (below 10 nM in human monocytes, corneal epithelial cells, or conjunctival fibroblasts) or below of relevant immune cells at or after 3 hours post dose. (Schopf, 2014, Vollmer, 2014). Surprisingly, the inventors have found that using a slightly higher concentration of roflumilast provides therapeutic coverage of 4× to 52× the IC50 for PDE4A, B, D in relevant immune tissues, even at 8 hours. This improvement allows for less frequent dosing, patient and physician convenience, and lower risk of side effects due to drug application.

In certain embodiments, the anterior ophthalmic disorder can be selected from the group consisting of but not limited to anterior uveitis, pan-uveitis; intermediate uveitis, uveitis associated with various autoimmune or systemic diseases including juvenile idiopathic arthritis (JIA), Behçet's disease, ankylosing spondylitis, sarcoidosis, Vogt-Koyanagi-Harada disease (VKH), lupus, rheumatoid arthritis (RA), multiple sclerosis or other autoimmune conditions; Idiopathic uveitis, HLA-B27 uveitis, uveitic glaucoma, iritis, iridocyclitis, rubeosis iritis, coloboma of the iris, Fuchs heterchromic iridocyclitis, iridocorneal endothelial syndrome (ICE), scleritis, corneal endothelial disease or dysfunction including endotheliitis, Fuch's corneal or endothelial dystrophy, endothelial corneal dysfunction associated with Graves ophthalmopathy or thyroid eye disease, megalocornea, Axenfeld's syndrome, Rieger's anomaly, Peters' anomaly or Anterior segment mesenchymal dysgenesis, ocular hypertension, glaucoma (including primary congenital, open angle, angle closure, normal tension, elevated episcleral venous pressure (EVP), Sturge Weber, or secondary glaucoma), bupthalmos, Posner Schlossman Syndrome or glaucomatocyclitic crisis, cataract, anterior post-surgical ocular inflammation, post-surgical ocular inflammation affecting corneal endothelium, deep corneal epithelial defects that also involve anterior segment dysfunction such as corneal endothelial dysfunction or neurotrophic keratitis, inflammation related to DSEK/DMET keratoplasty, rebound iritis, pseudoexfoliation syndrome, pupillary abnormalities, Stevens Johnson syndrome, Sjogren's syndrome, ocular graft-versus-host disease, ocular cicatricial pemphigoid, anterior sterile or infectious endophthalmitis, toxic anterior segment syndrome (TASS), sympathetic ophthalmia, hyphema, anterior segment ischemia, anterior chamber inflammation related to the underlying viral and other microbial causes, anterior chemical injury, and iatrogenic anterior chamber inflammation.

In preferred embodiments, the anterior chamber ophthalmic disorder is uveitis (anterior, pan, intermediate or posterior) or uveitis associated with HLA-B27, autoimmune or systemic disease (including but not limited to JIA, Behçet's, ankylosing spondylitis, VKH lupus, RA, and psoriatic arthritis), uveitic glaucoma, anterior or posterior chamber iatrogenic inflammation, ocular pemphigoid, or intraocular inflammatory disease of the corneal endothelium, iris, sclera, or choroid. The method can improve patient convenience and/or result in a reduction of at least one side effect relative to administration of a corticosteroid.

The methods disclosed herein are able to provide therapeutically effective amounts of roflumilast to the anterior chamber tissues, which are anatomically relevant for anterior ocular diseases such as anterior uveitis, uveitic glaucoma, or uveitis, iritis, or scleritis associated with autoimmune or systemic diseases. Further, these methods produce surprising results particularly in comparison to results suggested by early experiments and predictive modeling based on standard ophthalmic PK data sets and actual corneal exposures at 0.1%. Predictive modeling in the cornea, conjunctiva, ICB and AH, retina, choroid, and RPE suggested that the tissue levels of roflumilast should not increase dramatically at higher drug concentration and dose frequency, and would be much lower than observed and reported in the present application for anterior and vitreous compartments. As discovered by the inventors, the tissue concentration in relevant tissues to ocular inflammatory disease is surprisingly increased by an increase in both concentration and dose frequency.

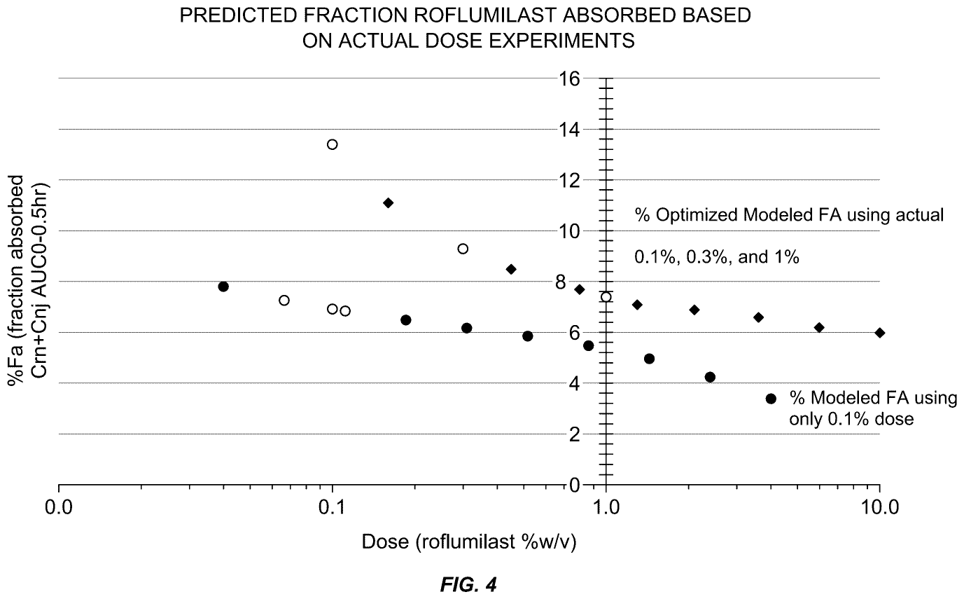

FIG. 4 provides data for the fraction of roflumilast absorbed (ng/g) in a rabbit cornea following exposure from an ophthalmic suspension of roflumilast, predicted by the concentration (% w/v) of roflumilast delivered to the ocular surface. Using the original model based solely on ophthalmic data and actual data for roflumilast ophthalmic suspension supplied to the ocular surface (0.1% w/v, BID) based on AUC 0-0.5 hrs, several sensitivity analyses were conducted to evaluate the effect of roflumilast dose in topical ophthalmic suspensions on various pharmacokinetic factors. This model was used to predict the impact of roflumilast on tissue concentrations at doses ranging between 0.05% to 10% w/v. The addition of actual data at higher dose concentrations (0.3% and 1%) and higher dose frequency (TID) show an increased level of absorption. The formulation in Examples 7 and 3 were used for model in FIG. 4.

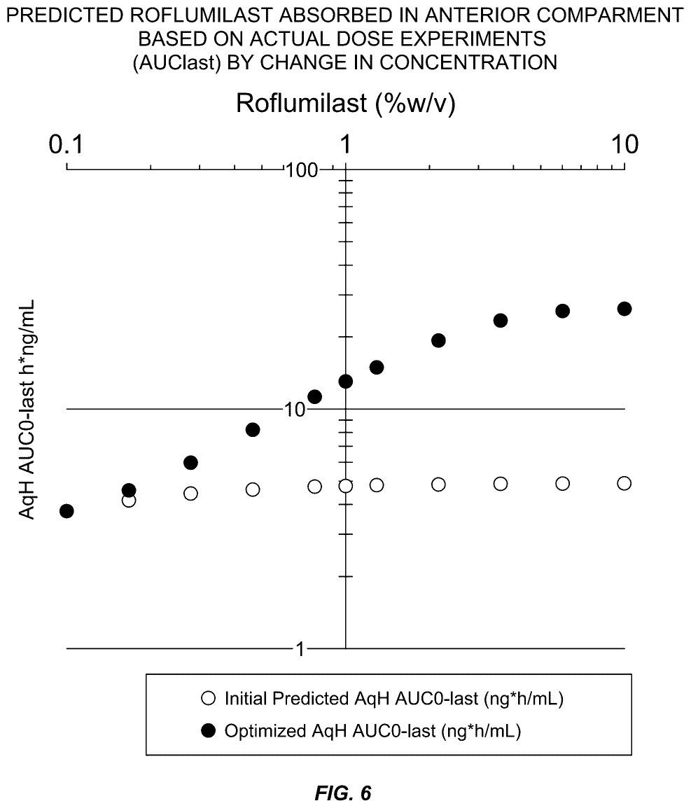

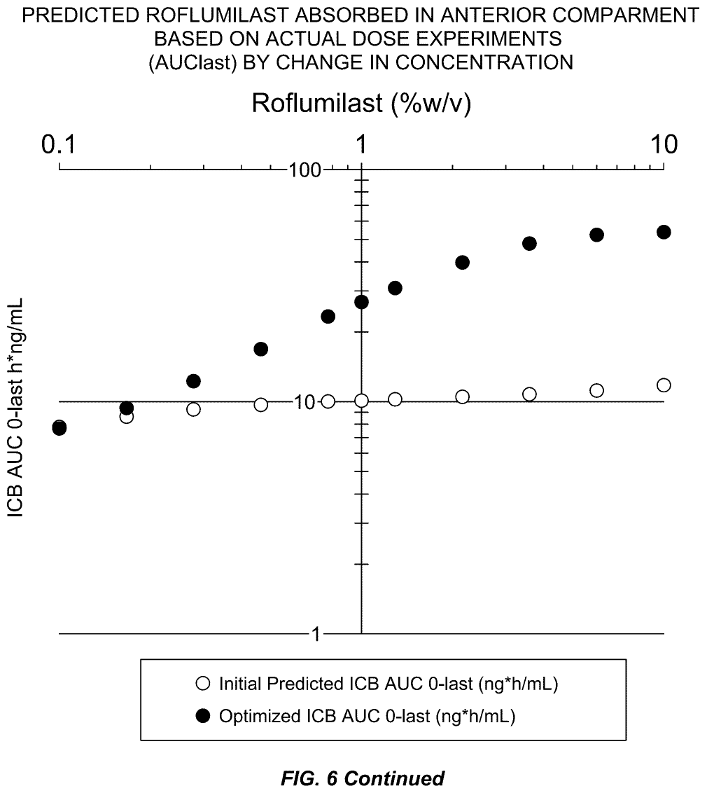

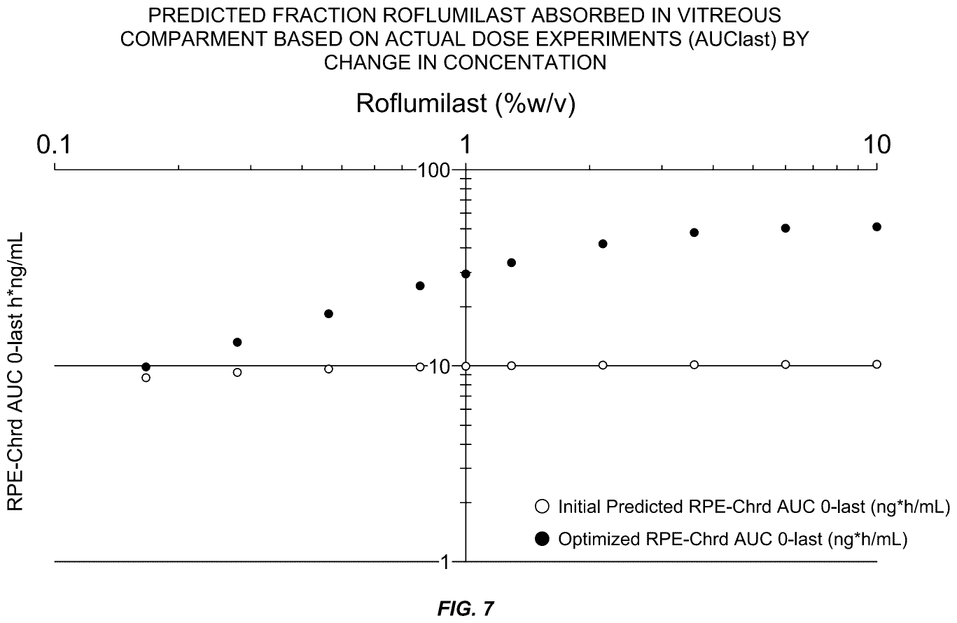

The predictive modeling is based on the relationship between the total dose in a drug product, intrinsic solubility of the drug substance, particle size of drug substance in suspensions, protein binding, and absorption in the eye and body. In particular, certain parameters can be predicted based on these known relations. For example, the fraction of a dose that is absorbed (% FA) into the cornea is a parameter that can be used to characterize absorption (which is examined in FIG. 4). Other commonly studied pharmacokinetic parameters include Cₘₐₓ and AUC (which is examined in FIGS. 6 and 7), which can be predicted within strict boundaries of a physiologically based absorption window. The surface-time concentration profile of roflumilast from topically applied ophthalmic suspensions (e.g., 99.95% solid particles and 0.05% dissolved fraction) are subject to rapid dilution and drainage by tears and nasolacrimal elimination, respectively. Using the roflumilast formulation set forth in Example 3 and the maximally absorbable dose (MAD) concept, a model was prepared predicting physicochemical and physiological limitations.

FIG. 4 provides a predictive model showing the predicted change in fraction of roflumilast (% FA) absorbed in the cornea as a function of the concentration of roflumilast (% w/v). In view of the model, formulating a more concentrated topical ophthalmic suspensions of roflumilast greater than 0.1% w/v was expected to result in diminishing efficiency in terms of % FA into the cornea, which will increase the fraction of that same dose entering the systemic circulation or lost via spillage (not shown). Actual data with higher concentration of active product as well as a more frequent dosing paradigm illustrate instead that it is possible to increase drug concentration to disease-relevant tissues in the anterior or posterior chambers.

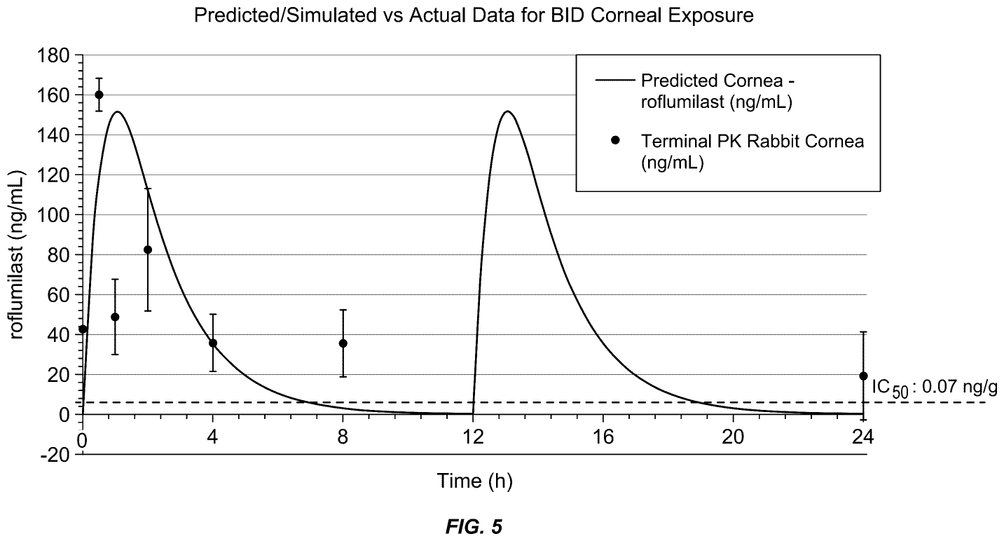

FIG. 5 illustrates the surprising finding that roflumilast topical ophthalmic suspension can provide a nearly 24 hour long-terminal level therapeutic concentrations of coverage of the relevant IC50 in ocular surface tissues such as the cornea, shown, compared to original modeled estimates which suggest a BID dosing would be required for 24 hour coverage.

FIGS. 6 and 7 provide a predictive model showing the predicted area under the curve (AUC) from 0-last (in this case, 24 hours) (ng/g) following administration of roflumilast as a function of the concentration of roflumilast (% w/v). If the relationship of an apparent maximal concentration reached (Cₘₐₓ) in corneal tissue at 30 min (apparent Tₘₐₓ) following topical ophthalmic instillation of a roflumilast ophthalmic suspension is examined as a function of various doses, the MAD concept would predict that due to the very low solubility and given particle size distribution of the drug, the most sensitive dose range on Cₘₐₓ would be less than 0.1. Original predictions suggested an expected saturation phenomenon where less-than proportional (sub-proportional) increase would be seen with higher doses.

No standardized methods exist for testing dissolution rates and absorption rates from topical ophthalmic space, hence the models were developed and validated. Computational modeling of topical ophthalmic roflumilast dosing was carried out using an ocular simulation computer program based on Matlab® (Mathworks, Natick, MA) programming language using a runtime Java® applet for a graphical user-friendly interface. Physiological and anatomic parameters for the ocular simulation were customized to model the pigmented rabbit eye, by entering appropriate volumes of distribution and surface areas of anterior and posterior compartment tissues. The physicochemical and physiological parameters of roflumilast used in the ocular simulation were its log P (estimated partition coefficient in an n-octanol/water 4.6), ocular pigment binding (estimated for the inventors at Covance, following the method by Koeberle et al. to be medium to high from equilibrium sepia melanin association), and systemic clearance following intravenous and oral administration. Formulation characteristics included were saturation concentration of 6 μg/mL, spherical suspension particles with an average radius of 4-5 micron, total dose potency of 1, 3, and 10 mg/mL), particle density of 1.2 g/mL, and simulated intrinsic dissolution rate. Based on these parameters, theoretical calculations were carried out for all simulations, while for matching concentrations with actual experimental data the model was used for comparison, fitting, and optimization.

In view of the predictive modeling, there was no expectation of substantially increased tissue concentrations in anterior and vitreous chamber ocular tissues with an increased concentration of roflumilast applied topically at a more frequent dosing interval; rather, the expectation would have been a dose saturation phenomenon based on the modeling studies and standard MAD curves. However, the inventors of the present application surprisingly discovered that roflumilast was able to provide more robust levels of roflumilast in various tissues of the eye at higher concentrations as a result of using the dosing regimens described herein including both higher concentration and frequency.

Additionally, in the aqueous humor (AH), multiple molecules do not reach high and sustained values, often due to solubility characteristics needed for AH durability, and high turnover of aqueous fluid, as described with steroids (Schopf 2014). Surprisingly, roflumilast had increasing levels of iris ciliary body (ICB) and AH distribution at these higher concentrations and frequencies. Secondarily, roflumilast's tendency to concentrate in highly vascular, immune rich tissue such as the conjunctiva fornix allows for “neighborhood” transverse transit to nearby scleral and ICB tissue, which are highly important for disease etiology in immune driven diseases due to immune cell concentration, particularly diseases driven by autoimmunity and Th17 and Th1.

In addition to providing unexpected efficacy, the methods disclosed herein can provide improved safety and reduced side effects. The safety profile of the methods disclosed herein can be similar to, equal to or better than the safety profile of frequently dosed, commonly used, and potent corticosteroids such as ophthalmic prednisolone suspensions which require a very high dose concentration and frequency to achieve clinically therapeutic levels. In certain embodiments, the methods result in a reduction of at least one side effect relative to administration of an ophthalmic prednisolone suspension or other corticosteroid. In certain embodiments, the reduced side effect is an ocular side effect selected from the group consisting of: increase of intraocular pressure, thinning of corneal, scleral and epithelial tissue, perforation of corneal, scleral and epithelial tissue, delayed or decreased wound or epithelial healing, hyperemia, lid edema, pain, ocular pruritis, urticaria, rash, allergic reactions, keratitis, conjunctivitis, posterior subcapsular cataract formation, glaucoma, optic nerve damage, corneal ulcers, mydriasis, defects in vision, burning, stinging, foreign body sensation, increased susceptibility to fungal, bacterial, or viral infections, reactivation of fungal or viral infections, masking of acute purulent infections, increased bleb formation after surgery, dry eye, punctate keratopathy, central serous chorioretinopathy, and ophthalmicus medicamentosa, loss of accommodation, ptosis, acute anterior uveitis and perforation of the globe.

In certain embodiments, the reduced side effect is a systemic side effect selected from the group consisting of: changes in blood glucose, weight gain or loss, decreased systemic wound healing, susceptibility to systemic microbial infections, irritation to tissues surrounding the eye, cold syndrome, pharyngitis, asthenia, back pain, headache, cough, nausea, rhinitis, sinusitis, osteoporosis, and taste perversion or dysgeusia, and sulfite-related anaphylaxis. Additionally, inactive ingredients and preservatives in these pharmaceutical ingredients can be absorbed into contact lenses of contact lens wearers.

Additionally, in certain embodiments, the use of a roflumilast pharmaceutical composition can provide the patient and caregiver benefit of less frequent administration which is both more convenient (allowing patients to work, travel, and leave the house without their medications) and also provides for avoidance of pain and discomfort of drug application many times per day. The methods disclosed herein allow for administration three or four times per day (TID or QID) compared to frequent dosing of eight times per day or more for corticosteroids at peak dosing. In some ocular diseases, physicians ask their patients to use a corticosteroid topical ophthalmic preparation once every one or two hours and wake during the night to instill additional doses, causing significant patient and caregiver burden. Steroid tapering regimens (starting at very high dose frequency, and tapering to a more convenient frequency over a period of weeks or months) are frequently used to provide both an increased convenience over time, but more importantly, a reduction in exposure to the side effects of high dose/high frequency steroids. However, this tapering can lead to rebound of inflammation, and a more convenient and sustainable administration which can provide a stable therapeutic dose is much preferred both clinically and conveniently. Often times, even the highest dose and frequency topical steroids do not provide enough therapeutic coverage of the anterior chamber or vitreous chamber tissues and patients may be placed onto either systemic, ocular injectable, or implanted ocular steroids, with a significant increase in severity and incidence of safety risks.

Additionally, for patients with moderate to long-term requirements for anti-inflammatory pharmaceutical intervention, whether because of chronic or recurrent disease, the use of corticosteroids comes with an increased patient and physician practice burden, as the patient will require frequent monitoring for safety concerns including intra-ocular pressure, cataract formation, and infection. This is particularly difficult in patients where this monitoring is challenging or uncomfortable: young children, elderly, and patients with ocular tissue which is sensitive due to long-term disease, all of which groups have frequent overlap with inflammatory ocular disease. In the current embodiment, this agent could avoid the need and cost for such frequent monitoring.

In the present invention, a patient in need thereof is administered an ophthalmic pharmaceutical composition comprising a therapeutically effective amount of roflumilast. The ophthalmic pharmaceutical composition can be formulated into such preparations utilizing a number of well-known and widely used methods to those of ordinary skill in the art. For example, the ophthalmic pharmaceutical composition can be a gel, ointment, cream, solution, suspension, or other topical formulation. In certain embodiments, the ophthalmic pharmaceutical composition can be a periocular or subconjunctival implant or injection via various sites (intravitreal, subconjunctival, sub-tenon, suprachoroidal, extraorbital, or others), or an intracorneal or intravitreal implant, injection, or depot, all designed to achieve delivery to anterior or vitreous chamber tissues. In preferred embodiments, the ophthalmic pharmaceutical composition is administered topically, directly to the eye, in the form of a suspension.

In certain embodiments, the ophthalmic pharmaceutical composition applied topically to the ocular surface can comprise roflumilast in a range from about 0.01% w/v to about 5.0% w/v, or from about 0.01% w/v to about 3.0% w/v, or from about 0.01% w/v to about 2.0% w/v, or from about 0.01% to about 1.0% w/v, or from about 0.01% to about 0.3% w/v, or from about 0.05% w/v to about 5.0% w/v, or from about 0.05% w/v to about 3.0% w/v, or from about 0.05% w/v to about 2.0% w/v, or from about 0.05% to about 1.0% w/v, or from about 0.05% to about 0.3% w/v, or from about 0.1% w/v to about 5.0% w/v, or from about 0.1% w/v to about 3.0% w/v, or from about 0.1% w/v to about 2.0% w/v, or from about 0.1% to about 1.0% w/v, or from about 0.1% to about 0.3% w/v, or from about 0.2% w/v to about 5.0% w/v, or from about 0.2% w/v to about 3.0% w/v, or from about 0.2% w/v to about 2.0% w/v, or from about 0.2% to about 1.0% w/v, or from about 0.2% to about 0.3% w/v, or from about 0.3% w/v to about 5.0% w/v, or from about 0.3% to about 3% w/v, or from about 0.3% to about 1% w/v. For example, the ophthalmic pharmaceutical comprises any of the following w/v percents of roflumilast: 0.01, %, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 7%, 1.8%, 1.9%, 1.0%, 2.1%, 2.2%, 2.3%, 2.4%, 2.5%, 2.6%, 2.7%, 2.8%, 2.9%, 3.0%, etc.

In certain embodiments, the ophthalmic pharmaceutical composition can be a suspension, solution eye drops, eye ointments, gels, creams, a spray, or another dosage form that allows for administration to the ocular surface for benefit in the anterior or vitreous chamber. In preferred embodiments, the pharmaceutical composition is a suspension, wherein the active ingredient (i.e., roflumilast) is suspended in a pharmaceutical carrier and/or excipients. In certain embodiments, the ophthalmic pharmaceutical composition of roflumilast comprises a viscosity agent, a surfactant, and a buffer. In certain embodiments, the ophthalmic pharmaceutical composition can include one or more additional excipients, including for example, a stabilizer, a preservative, a wetting agent, a diluting agent, a pH adjuster, a tonicity agent, or an absorption enhancer. In certain embodiments, the ophthalmic pharmaceutical composition can also be utilized for anterior, extraorbital, or vitreoretinal ophthalmic situations in the form of an injection (intravitreal, suprachoroidal, extraorbital or other), as a depot, an implantable adsorbent device for any ophthalmic or surrounding tissue placement, an in situ forming gel, or a drug/device combination, wherein the active ingredient (i.e., roflumilast) is suspended with one or more of the excipients above, for example a viscosity agent, a surfactant, or a buffer; with or without a device or inert depot compound, all designed to achieve delivery to anterior or vitreous chamber tissues.

In certain embodiments, the viscosity agent is at least one selected from the group consisting of hydroxypropyl methylcellulose (HPMC), hydroxyethylcellulose (HEC), polyvinyl pyrrolidione or povidone, carboxymethyl cellulose, hypromellose, methylcellulose, or polyvinyl alcohol (PVA). In certain embodiments, the viscosity agent is a dextran or gelatin. In addition, the viscosity agent can include a carbomer in certain embodiments, such as a carbomer copolymer Type A or a carbomer copolymer Type B including those marketed under the trade name Carbopol® by Lubrizol®. In certain embodiments, the ophthalmic pharmaceutical formulation can comprise a viscosity agent in a range from about 0.1% w/v to about 5.0% w/v, or about 0.1% w/v to about 4.5% w/v, or from about 0.1% w/v to about 4.0% w/v, or about 0.1% w/v to about 3.5% w/v, or from about 0.1% w/v to about 3.0% w/v, or about 0.1% w/v to about 2.5% w/v, or from about 0.1% w/v to about 2.0% w/v, or about 0.1% w/v to about 1.5% w/v, or from about 0.1% to about 1.0% w/v, or from about 0.1% to about 0.5% w/v. For example, the ophthalmic pharmaceutical comprises any of the following w/v percents of a viscosity agent: 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 7%, 1.8%, 1.9%, 1.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, etc.

In certain embodiments, the surfactant is at least one selected from the group consisting of polysorbates (including, polysorbate 20, polysorbate 40, polysorbate 60, and polysorbate 80) and tyloxapol. In certain embodiments, the ophthalmic pharmaceutical formulation can comprise a surfactant in a range from about 0.05% w/v to about 3.0% w/v, or from about 0.05% w/v to about 2.0% w/v, or from about 0.05% to about 1.0% w/v, or from about 0.05% to about 0.5% w/v, or from about 0.1% w/v to about 1.0% w/v, or from about 0.1% to about 0.5% w/v, or from about 0.1% to about 0.3% w/v, or from about 0.15% w/v to about 1.0% w/v, or from about 0.15% to about 0.5% w/v, or from about 0.15% to about 0.3% w/v. For example, the ophthalmic pharmaceutical comprises any of the following w/v percents of a surfactant: 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 7%, 1.8%, 1.9%, 1.0%, 2.1%, 2.2%, 2.3%, 2.4%, 2.5%, 2.6%, 2.7%, 2.8%, 2.9%, 3.0%, etc.

In certain embodiments, the buffer is at least one selected from the group consisting of citrate, phosphate, Tris-HCl (Tris), acetate, and borate buffers. In certain embodiments, the buffer can be any specific buffer in any specific concentration that maintains the pH of the composition as described herein. In certain embodiments, the ophthalmic pharmaceutical formulation can comprise a buffer in a range from about 0.2% w/v to about 7.5% w/v, or from about 0.2% w/v to about 5.0% w/v, or from about 0.2% to about 3.0% w/v, or from about 0.2% w/v to about 2.0% w/v, or from about 0.2% to about 1.0% w/v, or from about 0.3% w/v to about 7.5% w/v, or from about 0.3% w/v to about 5.0% w/v, or from about 0.3% to about 3.0% w/v, or from about 0.3% w/v to about 2.0% w/v, or from about 0.3% to about 1.0% w/v, or from about 0.5% w/v to about 7.5% w/v, or from about 0.5% w/v to about 5.0% w/v, or from about 0.5% to about 3.0% w/v, or from about 0.5% w/v to about 2.0% w/v, or from about 0.5% to about 1.0% w/v. For example, the ophthalmic pharmaceutical comprises any of the following w/v percents of a buffer: 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 7%, 1.8%, 1.9%, 1.0%, 2.1%, 2.2%, 2.3%, 2.4%, 2.5%, 2.6%, 2.7%, 2.8%, 2.9%, 3.0%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, etc.

In certain embodiments, the ophthalmic pharmaceutical formulation comprises 0.3% to 3.0% w/v of roflumilast, 0.3% to 3.0% w/v hydroxypropyl methylcellulose (HPMC), 0.01% to 0.5% w/v of a surfactant selected from the group consisting of a polysorbate and tyloxapol, 0.05% to 1% of a buffer, and 0.1% to 1% of a tonicity agent. In certain embodiments, the ophthalmic pharmaceutical formulation comprises 0.3% to 3.0% w/v of roflumilast, 0.3% to 3.0% w/v hydroxyethylcellulose (HEC), 0.01% to 0.5% w/v of a surfactant selected from the group consisting of a polysorbate and tyloxapol, 0.05% to 1% of a buffer, and 0.1% to 1% of a tonicity agent. In certain embodiments, the ophthalmic pharmaceutical formulation comprises 0.3% to 3.0% w/v of roflumilast, 0.3% to 3.0% w/v polyvinyl pyrrolidione or povidone, 0.01% to 0.5% w/v of a surfactant selected from the group consisting of a polysorbate and tyloxapol, 0.05% to 1% of a buffer, and 0.1% to 1% of a tonicity agent. In certain embodiments, the ophthalmic pharmaceutical formulation comprises 0.3% to 3.0% w/v of roflumilast, 0.3% to 3.0% w/v a carbomer copolymer Type A or a carbomer copolymer Type B, 0.01% to 0.5% w/v of a surfactant selected from the group consisting of a polysorbate and tyloxapol, 0.05% to 1% of a buffer, and 0.1% to 1% of a tonicity agent.

In certain embodiments, the ophthalmic pharmaceutical formulation is an ointment. The ointment can include inactive ingredients selected from the group consisting of petrolatum, mineral oil. In such embodiments, the ophthalmic pharmaceutical formulation can comprise a therapeutically effective amount of roflumilast, petrolatum, and mineral oil. In certain embodiments, the composition comprises from about 0.1% w/v to about 3.0% w/v, or from about 0.1% w/v to about 2.0% w/v, or from about 0.1% to about 1.0% w/v, or from about 0.25% w/v to about 3.0% w/v, or from about 0.25% w/v to about 2.0% w/v, or from about 0.25% to about 1.0% w/v of roflumilast, from about 0.5% w/v to about 3.0% w/v, or from about 0.5% w/v to about 2.0% w/v, or from about 0.5% to about 1.0% w/v of roflumilast. In certain embodiments, the composition comprises from about 75% to about 85% w/w of petrolatum, or more preferably from about 75% to about 80% w/w of petrolatum. In certain embodiments, the composition comprises from about 15% to about 25% w/w mineral oil, or more preferably from about 15% to about 20% w/w of mineral oil. The ointment can provide benefits relative to suspensions, including for example, increasing contact time and increasing the soluble drug concentration in the dosing system, which can be important for a water-insoluble drug like roflumilast.

Roflumilast can undergo hydrolysis in certain ophthalmic pharmaceutical compositions and under certain standard sterile manufacturing processes. In certain embodiments, the pH of the ophthalmic pharmaceutical composition is between 5.5 and 7.5. In preferred embodiments, the pH of the ophthalmic pharmaceutical compositions is between about 6.0 and about 6.7 to reduce the rate of hydrolysis of roflumilast. In certain embodiments, the pH of the ophthalmic pharmaceutical composition is between about 6.2 and about 6.7. In certain embodiments, the osmolality of the ophthalmic pharmaceutical composition is about 270 mOsm/kg to 330 mOsm/kg, more preferably about 270 mOsm/kg to about 300 mOsm/kg, and even more preferably 270 mOsm/kg to 280 mOsm/kg.

The ophthalmic pharmaceutical compositions are preferably stable and exhibit a particle size distribution suitable for ophthalmic delivery. Particle size of the ophthalmic pharmaceutical composition for suspensions can be assessed using laser diffraction methods. Laser diffraction is recognized by standards and guidance agencies including ISO and ASTM and is widely used to determine particle size distributions. In conducting the assessment, the sample is passed through a laser beam, which results in laser light scattered at a range of angles. Detectors placed at fixed angles measure the intensity of light scattered at that position. A mathematical model is then applied to generate a particle size distribution.

In particle size determinations, the median value is defined as the value where half of the population resides above this point, and half resides below this point. For particle size distributions, the median is called the D50. The D50 is the size that splits the distribution with half above and half below this diameter. The distribution width may also be characterized by citing one, two or three values on the x-axis, typically some combination of the D10, D50, and D90. The D50 (or the median), as discussed above, refers to the diameter wherein half of the population lies below this value. Similarly, 90 percent of the distribution lies below the D90, and 10 percent of the population lies below the D10.

In certain embodiments of the present invention, the ophthalmic pharmaceutical composition exhibits a particle size distribution characterized by a d90 value of less than or equal to about 50 μm prior to preferential processing. In certain embodiments, the ophthalmic pharmaceutical composition exhibits a particle size distribution characterized by a d90 value of from about 5 μm to about 25 μm. In certain embodiments, the pharmaceutical compositions exhibits a particle size distribution characterized by a d90 value of from about 5 μm to about 15 μm. In preferred embodiments, the pharmaceutical compositions exhibit a particle size distribution characterized by a d90 value of less than or equal to 10 μm.

In certain embodiments, the pharmaceutical composition of roflumilast is sterilized using slow dry heat sterilization at a temperature less than the melting point of roflumilast, gamma radiation, or other methods of sterilization. In certain embodiments, gamma radiation or other terminal product sterilization methods can be used to sterilize the final drug product in its terminal packaging by application of low-moderate level gamma radiation to ensure sterility, which is a preferred embodiment for maximum patient safety and comfort, as the product will not need to contain preservatives which are known to sting upon application. In certain embodiments, the ophthalmic pharmaceutical composition can be characterized by a retained potency of greater than 99% of the original value of active substances. In certain embodiments, the retained potency is greater than 99.1%, 99.2%, 99.3%, 99.4%, or 99.5% of the original value of active substances.

The following examples illustrate certain embodiments of the invention without limitation.

### EXAMPLES

While various embodiments have been described herein, it should be understood that they have been presented by way of example only, and not limitation. Thus, the breadth and scope of the present disclosure should not be limited by any of the described exemplary embodiments. Moreover, any combination of the above-described elements in all possible variations thereof is encompassed by the disclosure unless otherwise indicated herein or otherwise clearly contradicted by context.

### Example 1

The ophthalmic pharmaceutical compositions comprising roflumilast set forth in Table 1 were prepared.

TABLE 1
Ophthalmic Pharmaceutical Suspensions of Roflumilast

| | 0.1% | 0.3% | 1.0% | 3.0% |
|---|---|---|---|---|
| | Roflumilast | Roflumilast | Roflumilast | Roflumilast |
| | HPMC | HPMC | HPMC | HPMC |
| Ingredient | Suspension | Suspension | Suspension | Suspension |
| Roflumilast | 0.1% w/w | 0.3% w/w | 1.0% w/w | 3.0% w/w |
| Hypromellose | 0.3% | 0.3% | 0.3% | 0.3% |
| (HPMC) | | | | |
| Polysorbate 80 | 0.1% | 0.1% | 0.1% | 0.1% |
| Sodium | 0.25% | 0.25% | 0.25% | 0.25% |
| phosphate | | | | |
| dibasic | | | | |
| heptahydrate | | | | |
| Citric acid | 0.05% | 0.05% | 0.05% | 0.05% |
| monohydrate | | | | |
| Sodium chloride | 0.7% | 0.7% | 0.7% | 0.7% |
| 1N HCl and/or | Adjust pH to 6.5 | Adjust pH to 6.5 | Adjust pH to 6.5 | Adjust pH to 6.5 |
| 1N NaOH | | | | |
| Purified Water | q.s. to 100% | q.s. to 100% | q.s. to 100% | q.s. to 100% |

### Example 2

The ophthalmic pharmaceutical compositions comprising roflumilast set forth in Table 2 was prepared.

TABLE 2
Ophthalmic Pharmaceutical Suspension of Roflumilast

| Ingredient | % w/v |
|---|---|
| Roflumilast | 0.1% w/v |
| Hydroxyethyl cellulose | 0.35% w/v |
| Polysorbate 80 | 0.1% w/v |
| Phosphate/Citrate Buffer | 0.45%/0.05% |
| Water for injection | q.s. ad 1.0 mL |

### Example 3

The ophthalmic pharmaceutical compositions comprising roflumilast set forth in Table 3 were prepared.

TABLE 3
Ophthalmic Pharmaceutical Suspensions of Roflumilast

| | 0.1% | 0.3% | 1.0% | 3.0% |
|---|---|---|---|---|
| | Roflumilast | Roflumilast | Roflumilast | Roflumilast |
| Ingredient | PVP Suspension | PVP Suspension | PVP Suspension | PVP Suspension |
| Roflumilast | 0.1% w/w | 0.3% w/w | 1.0% w/w | 3.0% w/w |
| Povidone (K30) | 0.6% | 0.6% | 0.6% | 0.6% |
| PVP | | | | |
| Tyloxapol | 0.3% | 0.3% | 0.3% | 0.3% |
| Sodium | 0.25% | 0.25% | 0.25% | 0.25% |
| phosphate | | | | |
| dibasic | | | | |
| heptahydrate | | | | |
| Citric acid | 0.05% | 0.05% | 0.05% | 0.05% |
| monohydrate | | | | |
| Sodium chloride | 0.7% | 0.7% | 0.7% | 0.7% |
| 1N HCl and/or | Adjust pH to 6.5 | Adjust pH to 6.5 | Adjust pH to 6.5 | Adjust pH to 6.5 |
| 1N NaOH | | | | |
| Purified Water | q.s. to 100% | q.s. to 100% | q.s. to 100% | q.s. to 100% |

### Example 4

The ophthalmic pharmaceutical composition comprising roflumilast set forth in Table 4 was prepared.

TABLE 4
Ophthalmic Pharmaceutical Suspension of Roflumilast

| Ingredient | % w/v |
|---|---|
| Roflumilast | 0.1% w/v |
| Carboxymethyl cellulose | 0.5% w/v |
| Polysorbate 80 | 0.1% w/v |
| Phosphate/Citrate Buffer | 0.45%/0.05% |
| Water for injection | q.s. ad 1.0 mL |

### Examples 5 and 6

The ophthalmic pharmaceutical ointments comprising roflumilast set forth in Table 5 was prepared.

TABLE 5
Ophthalmic Pharmaceutical Ointments of Roflumilast

| | Example 5 | Example 6 |
|---|---|---|
| Ingredient | (% w/w) | (% w/w) |
| Roflumilast | 0.1% w/w | 1% w/w |
| Mineral oil | 20.2% w/w | 20% w/w |
| Petrolatum | 79.7% w/w | 79% w/w |

### Example 7

An ophthalmic pharmaceutical compositions comprising roflumilast as set forth in Table 6 was prepared.

TABLE 6
Ophthalmic Pharmaceutical Suspension of Roflumilast at 0.1% w/v

| Excipient | Concentration (mg/mL) |
|---|---|
| Roflumilast | 1.0 |
| Carbopol 974B (Lubrizol) | 2.5 |
| Tyloxapol | 0.5 |
| Sodium chloride | 3.0 |
| Mannitol | 3.0 |
| Propylene Glycol | 14.0 |
| NaOH/HCl | As needed to adjust pH to 7.4 |

### Example 8

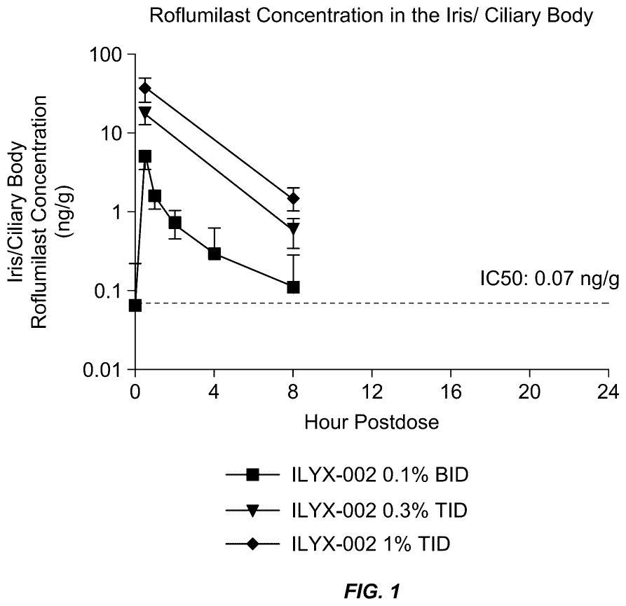

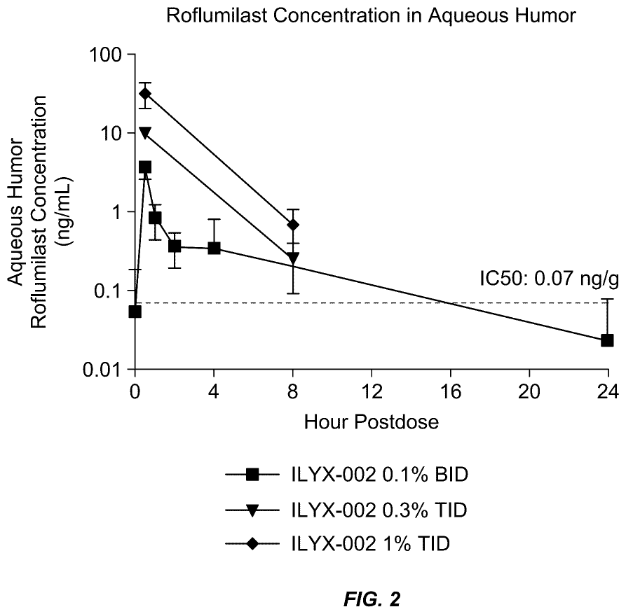

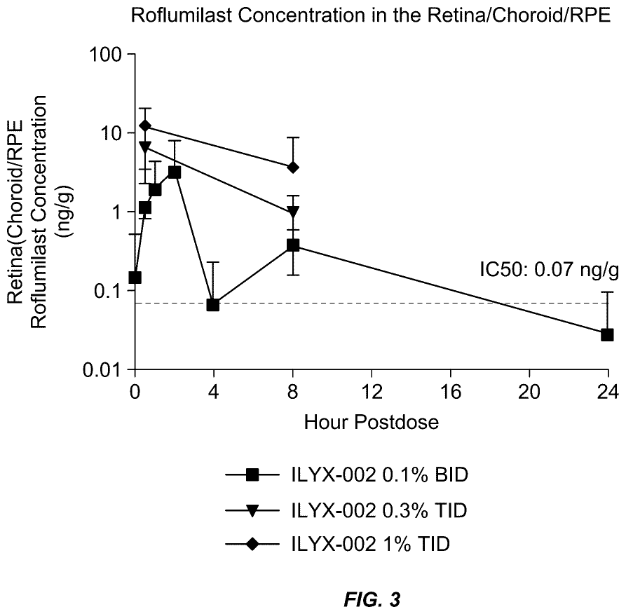

Two unique animal studies were conducted to assess the concentration of roflumilast in various tissues following administration of different concentrations of Example 3. The concentration in various tissues was assessed following five days of administration of the following dosing regimens: (a) 1% w/v roflumilast three times a day (TID); (b) 0.3% w/v roflumilast three times a day (TID); or (c) 0.1% w/v roflumilast two times a day (BID). In one study encompassing (a) and (b), n=6 Dutch belted rabbits per dose arm were dosed topically three times per day according to arm on days 1 to 4, and on day 5, a single dose was given at hour 0. In a different study with the same methodologies and practices, n=21 Dutch belted rabbits represented in group (c) were dosed two times per day on days 1 to 4, and on day 5, a single dose was given at hour 0. In both studies, under all relevant industry standards and guidelines, 3 animals per time point were euthanized and tissues were collected for bioanalytics at the marked time points: for (a) and (b), at 0.5 hours and 8 hours post dose. In the original study conducted solely with arm (c), a dose curve was created using time points at 0.5, 2, 4, 8, and 24 hours. To reduce the number of animals in the second experiment, for group (a) and (b), only 0.5 and 8 hours were tested. FIG. 1 provides data for the concentration of roflumilast (ng/g) in the iris ciliary body (ICB) as a function of time following: (a) 1% w/v roflumilast administered three times a day; (b) 0.3% w/v roflumilast administered three times a day; or (c) 0.1% w/v roflumilast administered two times a day. FIG. 2 provides data for the concentration of roflumilast (ng/g) in the aqueous humor (AH) as a function of time following: (a) 1% w/v roflumilast administered three times a day; (b) 0.3% w/v roflumilast administered three times a day; or (c) 0.1% w/v roflumilast administered two times a day. FIG. 3 provides data for the concentration of roflumilast (ng/g) in the retina/choroid/retina pigment epithelium (RPE) as a function of time following: (a) 1% w/v roflumilast administered three times a day; (b) 0.3% w/v roflumilast administered three times a day; or (c) 0.1% w/v roflumilast administered two times a day.

Tissue levels of roflumilast were increased with higher doses (1%>0.3%>0.1%) and treatment frequency (three times a day (TID) vs. two times a day (BID)) in the ICB, AH, and retina/choroid/RPE. In the ICB, AH, and retina/choroid/RPE, both 0.3% and 1% provide strong elevation in tissue levels above the IC50 level.

### Example 9

When comparing the therapeutic potential of roflumilast topical ophthalmic suspension to other potential therapeutic agents for treatment of ocular diseases beyond the ocular surface, it is critical not only to ensure that the agent is safe, but also that there is a reasonable potential that the relevant ocular tissues receive and maintain an adequate drug coverage level to maintain a therapeutic effect. When attempting to cover an enzymatic target such as PDE4, it is important to not only reach the IC50, or the point at which the therapeutic agent is reaching half of the maximal inhibitory concentration, but also to provide tissue coverage beyond that point. The metric of reaching 10× the ratio of the IC50 at Cmax is a rule of thumb to understand whether a substance would be active in inhibiting a target. At the point at which the therapeutic agent would be re-dosed, it is important that the therapeutic agent still have coverage above the IC50. Given the difficulty in reaching the anterior chamber and the relevant tissues for immune trafficking of t-cells, it is common that the concentration of any topically dosed agent will be higher in the ocular surface tissues (for example, cornea, conjunctiva) than in anterior tissues (for example, ICB, AH), and very much higher than vitreous chamber or vitreo-retinal tissues (for example, retina/choroid). The inventors were surprised to find that unlike in the predictive models which would suggest that increasing dose frequency and concentration would yield a lower maximally absorbable dose and peak tissue level threshold with rapid loss of tissue roflumilast levels in the anterior and vitreous chambers, increasing both concentration and frequency of dose (1) lifted drug concentration of roflumilast to ratios above IC50 of PDE4 to ratios considered to be highly therapeutic, (2) yielded higher AUC than expected for Roflumilast in anterior and vitreal chambers, and (3) Roflumilast concentrations remained elevated in the tissues longer than expected.

Table 7 compares the data from FIGS. 1, 2, and 3, to the IC50 of t-cells for the PDE4 A, B, D targets (0.07 ng/ml or 0.07 ng/g), illustrating the fold-coverage of IC50 at 0.5 and 8 hrs in the tissues as increased by concentration and frequency of roflumilast topical ocular dosing. Table 7 shows the ratios of roflumilast tissue concentration in either ng/ml or ng/g to IC50 for the cornea, an ocular surface tissue, ICB and AH, anterior tissues, and Retina/Choroid, a vitreous tissue, by dose concentration. The IC50 for roflumilast to PDE4, given its extreme potency and selectivity, is 0.07 nm/g or nm/ml or less. (Claveau, 2004) This ratio is shown at both 0.5 hours, closest to a Cmax time point, and 8 hrs, which would be a time point close to a redosing time point for a TID dosing. The ideal range is 10×-100× for enzymatic blockade at Cmax (0-1 hr), 5-10× at Tlast (depending on dose frequency).

TABLE 7

| | Comparison of Coverage Ratios Over IC50 | | | | | |
|---|---|---|---|---|---|---|
| | By Dose: Ratio of Roflumilast Concentration | | | | | |
| | (PDE4 IC50 ng/ml) | | | | | |
| | 0.10% | | 0.30% | | 1.00% | |
| | 0.5 hrs | 8 hrs | 0.5 hrs | 8 hrs | 0.5 hrs | 8 hrs |
| Cornea | 2286× | 509× | — | — | — | — |
| ICB | 66× | 2× | 247× | 9× | 534× | 22× |
| AH | 53× | 0× | 143× | 4× | 476× | 10× |
| RET/CHOR | 15× | 5× | 91× | 14× | 167× | 52× |

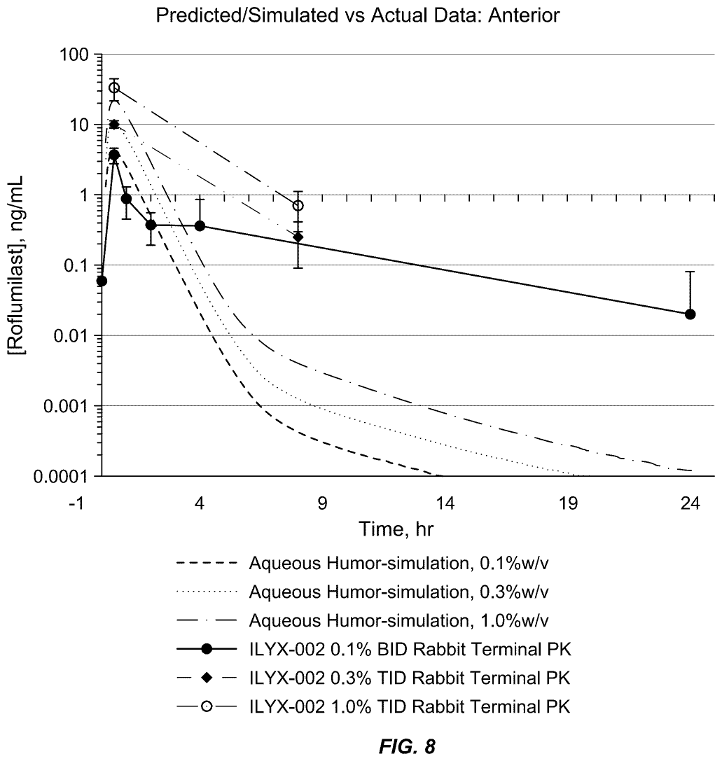

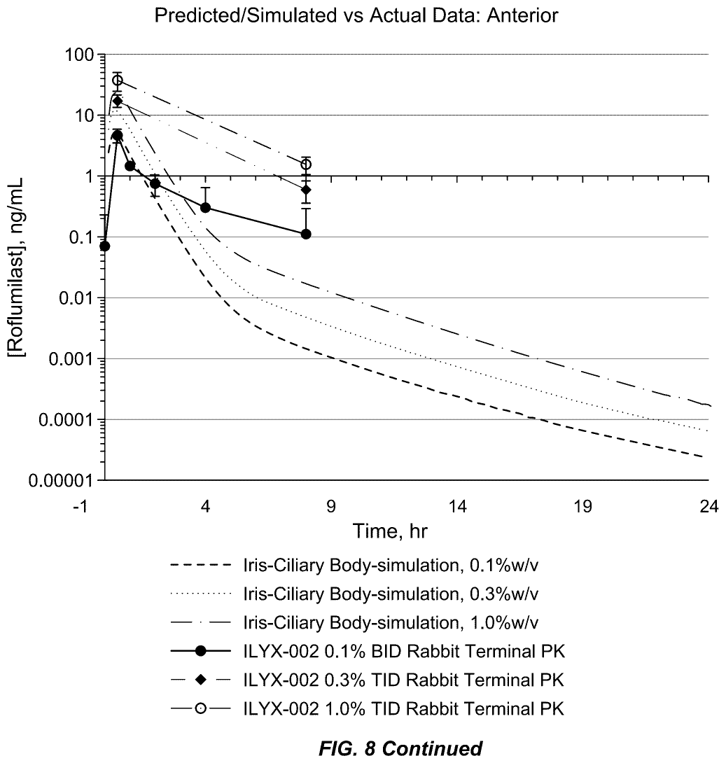

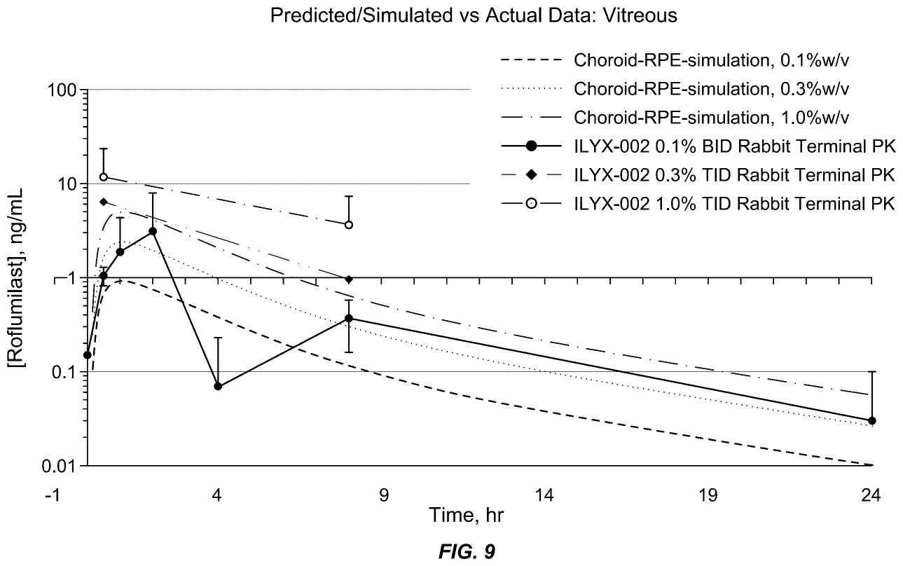

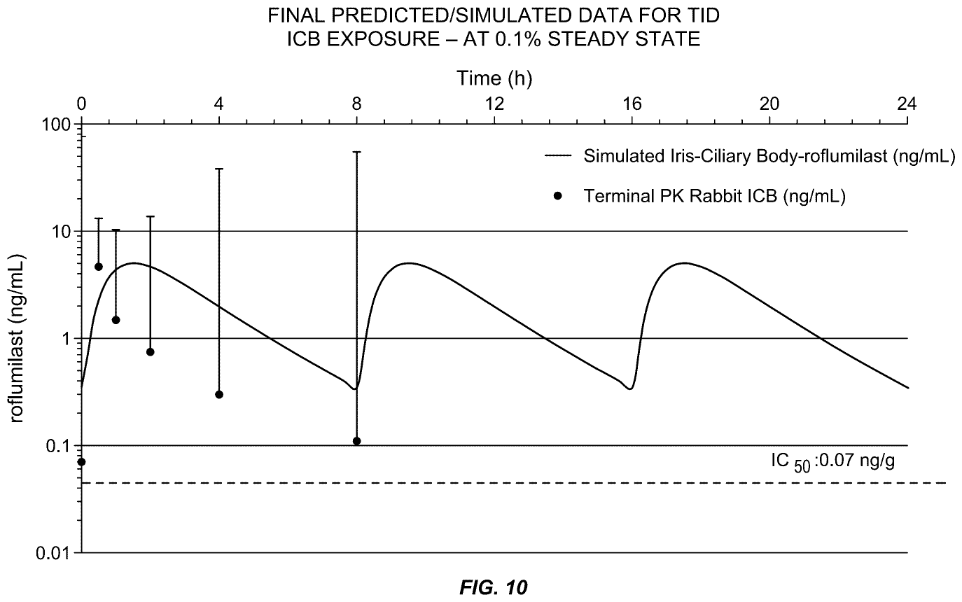

It would be expected that the ratios of ocular surface would be highest of all, and that is seen in the data in Table 7, given topical delivery. It is surprising that given the results of the predictive models shown in FIGS. 8, 9, and 10, that the anterior tissues ratio increase with higher dose concentration and frequency to high therapeutic levels at the 0.3% dose, and even higher levels for the 1% dose, representing therapeutically meaningful coverage. In FIG. 8, the actual concentration of roflumilast at 0.5 and 8 hours in these disease-relevant tissues is surprisingly higher than the original modeled estimate for the concentration of roflumilast (ng/g) in the ICB and AH as a function of time. In FIG. 9, the actual concentration of roflumilast at 0.5 and 8 hours in these disease-relevant tissues is surprisingly higher than the original modeled estimate for the concentration of roflumilast (ng/g) in the retina/choroid/RPE as a function of time. In FIG. 10, the simulation suggests that if the roflumilast suspension were given three times per day at 0.1% TID, rather than the 0.1% BID (shown in the actual terminal PK), it could provide therapeutic level of roflumilast coverage of the IC50 of relevant PDE4 targets. Not shown is modeling which would suggest that the tissue levels would be even higher and long-lasting assuming both the increased frequency of dosing and the increased concentration. It is surprising given the fraction absorbed that the retina/choroid tissues also see meaningfully therapeutic levels of drug ratio to IC50, as retina and choroid tissues are seen as very difficult to reach with topical treatment.

### Example 10

A study was conducted to evaluate clinical, ophthalmic, histopathology, and toxokinetic outcomes of repeated topical ocular administration of roflumilast. In the study, male and female dogs (n=3M/3F per arm to week 13, 2M/2F to additional 4 week recovery) received 13 weeks of topical treatment, three times a day, followed by a four week recovery period, in four dose arms, 0%, 0.1%, 0.3%, and 1% concentration of roflumilast. In addition to regular safety and well-being checks, animals were given regular ophthalmic exams, measurement of weight, tonometry to test intraocular pressure, pachymetery to test corneal thickness, clinical chemistry and hematological evaluation, and other routine checks of health and well-being. At the point of study termination, ocular tissues and major organs were tested for macro- and microscopic observation. There were no major test article related findings on major clinical or pathological findings in life or post-mortem, and the therapeutic agents were considered well-tolerated. Male and female beagle dogs (n=5 per group) were administered: (a) vehicle-based placebo three times a day; (b) a 0.1% w/v roflumilast formulation (Example 3) three times a day; (c) a 0.3% w/v roflumilast formulation (Example 3) three times a day; or (d) a 1.0% w/v roflumilast formulation (Example 3) three times a day. The study lasted for 84 days of active treatment and 28 days of non-treatment follow up.

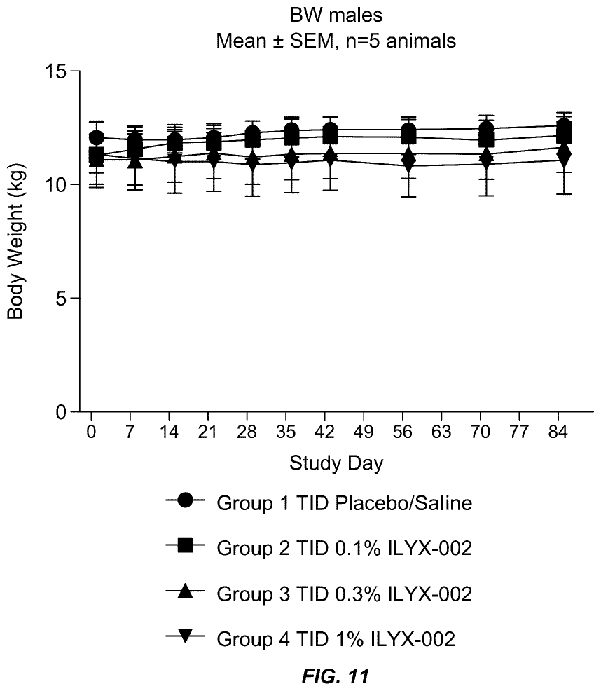

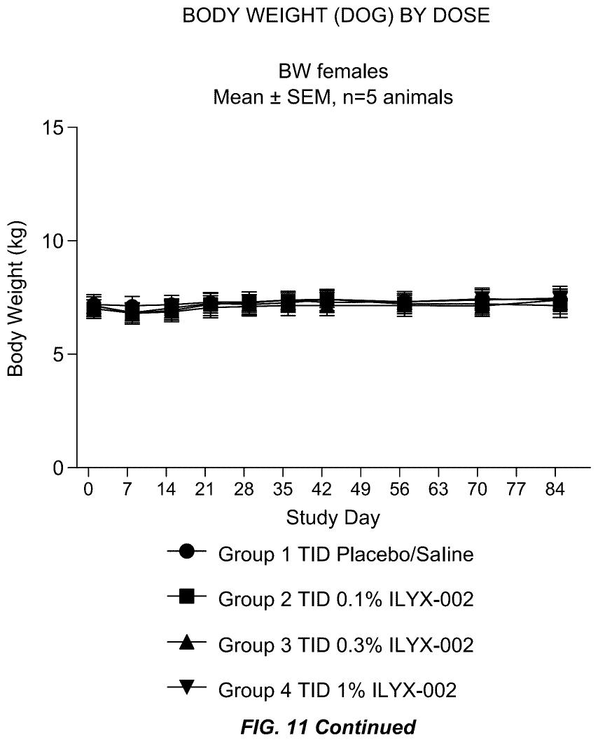

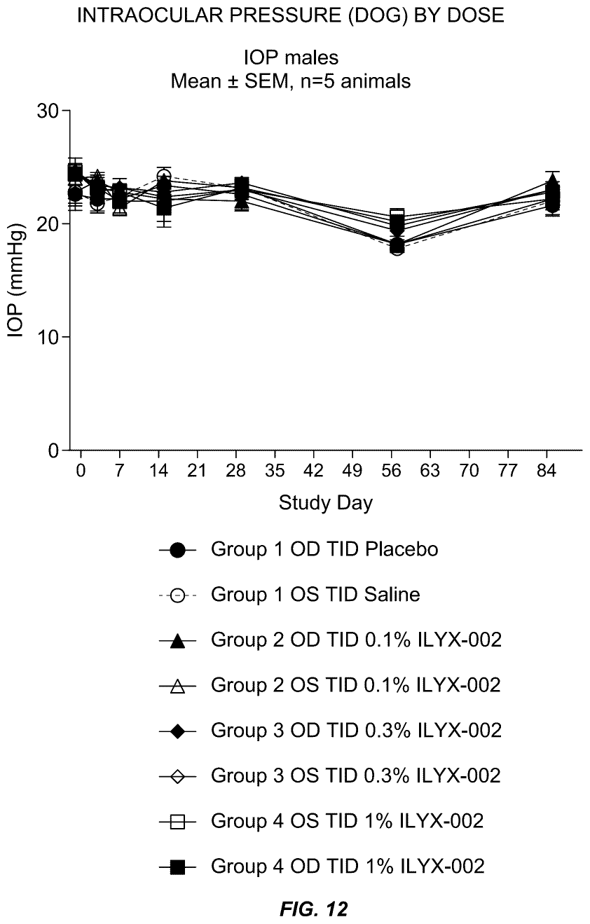

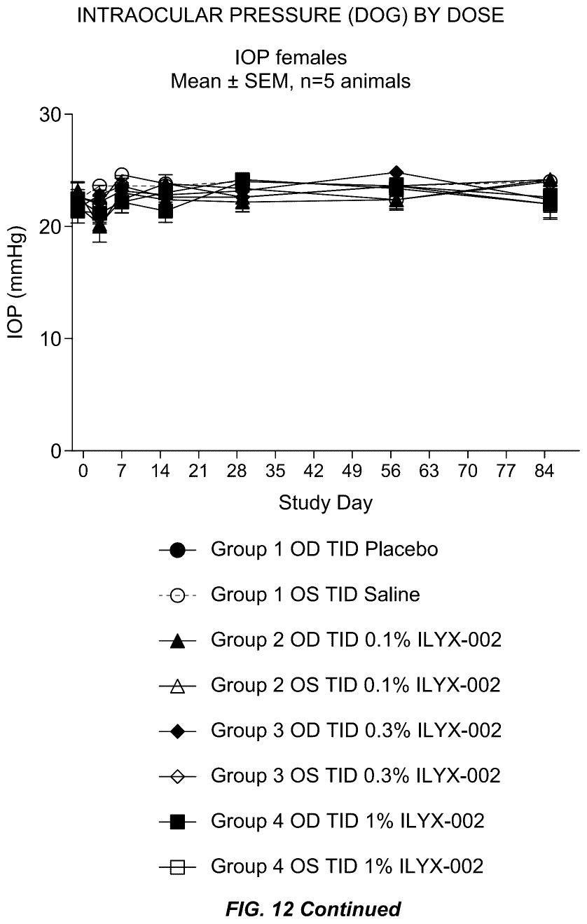

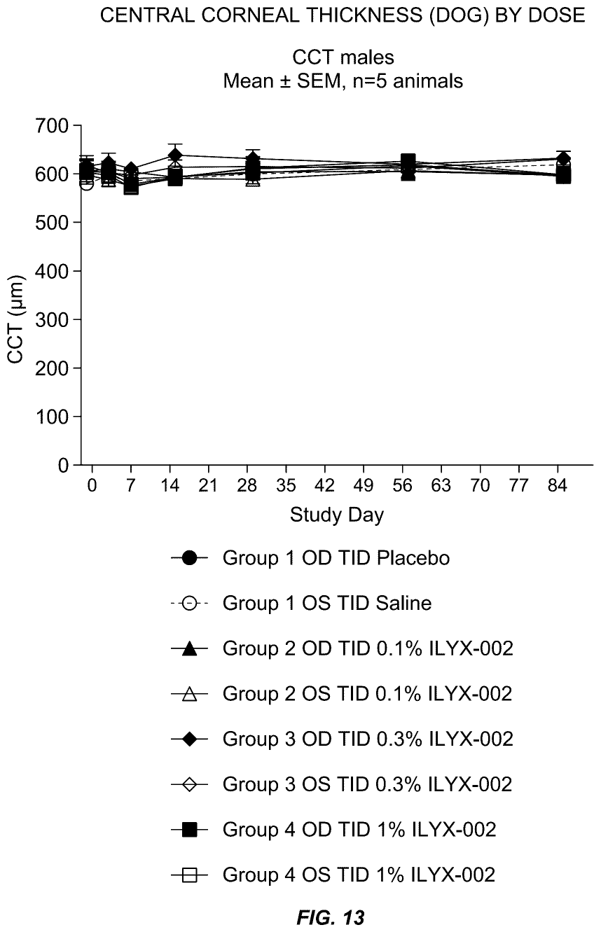

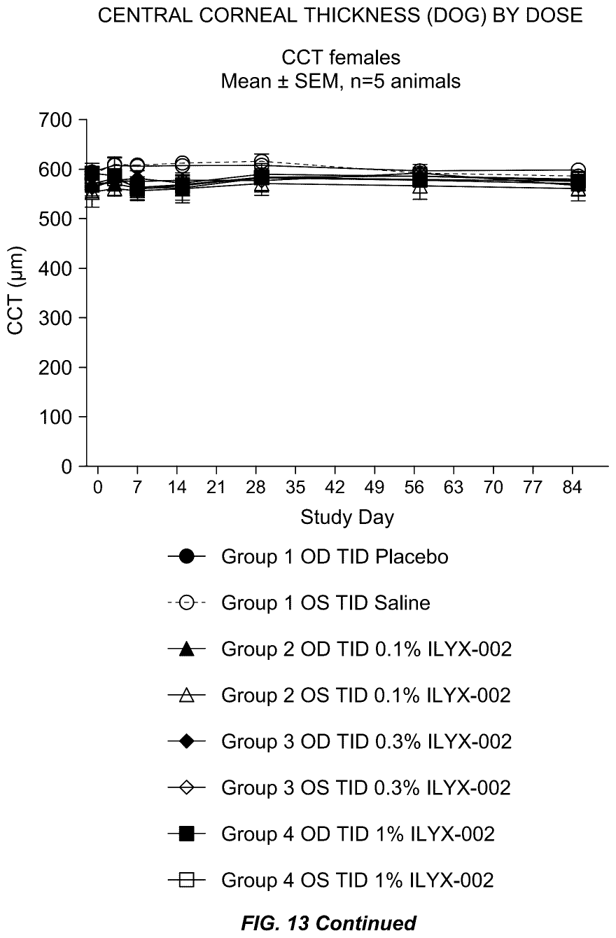

FIGS. 11, 12, and 13 provide the results of the study. Parameters shown in the figures as exemplary of the results included body weight, intraocular pressure (IOP), and central corneal thickness (CCT). FIG. 11 provides data for the clinical response of body weight. FIG. 12 provides data for the clinical response of intraocular pressure FIG. 13 provides data for the clinical response of central corneal thickness. As illustrated in FIGS. 11, 12, and 13, the formulation of roflumilast was well tolerated at all concentrations. FIG. 11 illustrates that for all concentrations of roflumilast the dogs did not lose weight over the course of the study as compared to vehicle-based placebo. FIG. 12 illustrates that the intraocular pressure was substantially the same for all treatment groups with only transient changes seen sporadically in the population. FIG. 13 illustrates that the central corneal thickness was substantially consistent for all treatment groups over the duration of the study. Ophthalmic and other exams also showed that the drug was well tolerated and had little to no systemic adverse effect on the eye or other organs of the body. Thus, given the wide range of concentrations tested, it is expected that all concentrations of roflumilast, including those yielding higher and longer duration tissue concentrations in the anterior and vitreous chambers, would be well tolerated and produce reduced side effects relative to other standard of care treatments currently available.

### Example 11

A study was conducted to evaluate toxicity outcomes of repeated topical ocular administration of roflumilast. Male and female New Zealand white rabbits (n=7 per group) were administered: (a) vehicle-based placebo three times a day; (b) a 0.1% w/v roflumilast formulation (Example 3) three times a day; (c) a 0.3% w/v roflumilast formulation (Example 3) three times a day; or (d) a 1.0% w/v roflumilast formulation (Example 3) three times a day. In the study, male and female New Zealand White rabbits received 13 weeks of topical treatment, three times a day, followed by a four-week recovery period, in four dose arms, 0%, 0.1%, 0.3%, and 1% concentration of roflumilast. In addition to regular safety and well-being checks, animals were given regular ophthalmic exams, measurement of weight, tonometry to test intraocular pressure, clinical chemistry and hematological evaluation, and other routine checks of health and well being. At the point of study termination, ocular tissues and major organs were tested for macro- and microscopic observation. There were no major test article related findings on major clinical or pathological findings in life or post-mortem, and the therapeutic agents were considered well-tolerated. The study lasted for 84 days of active treatment and 28 days of non-treatment follow up. Parameters evaluated included body weight, intraocular pressure (IOP), and central corneal thickness (CCT).

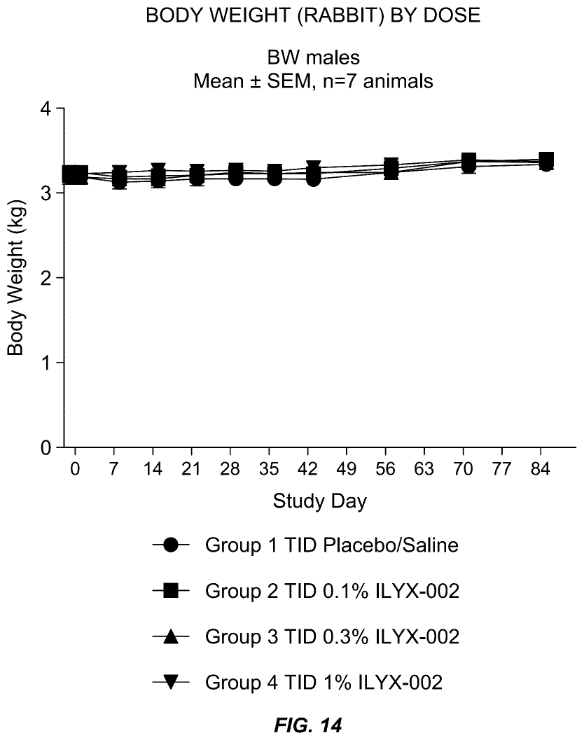

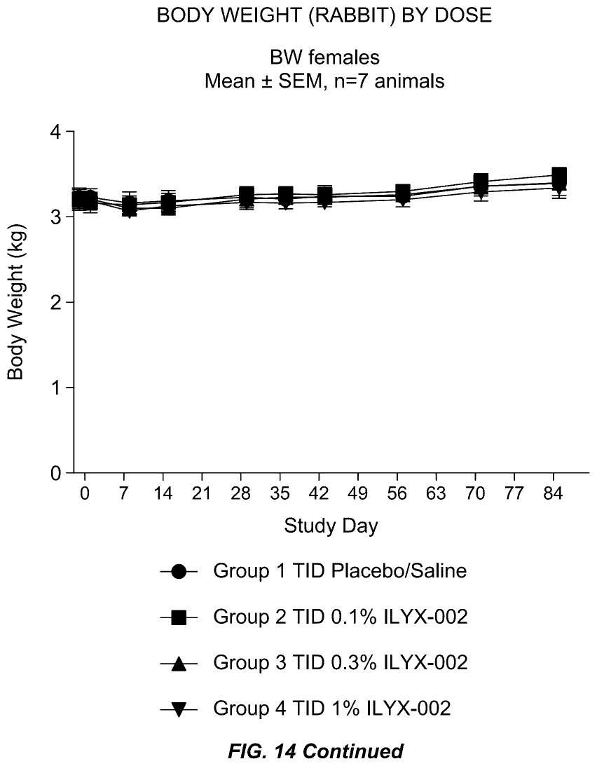

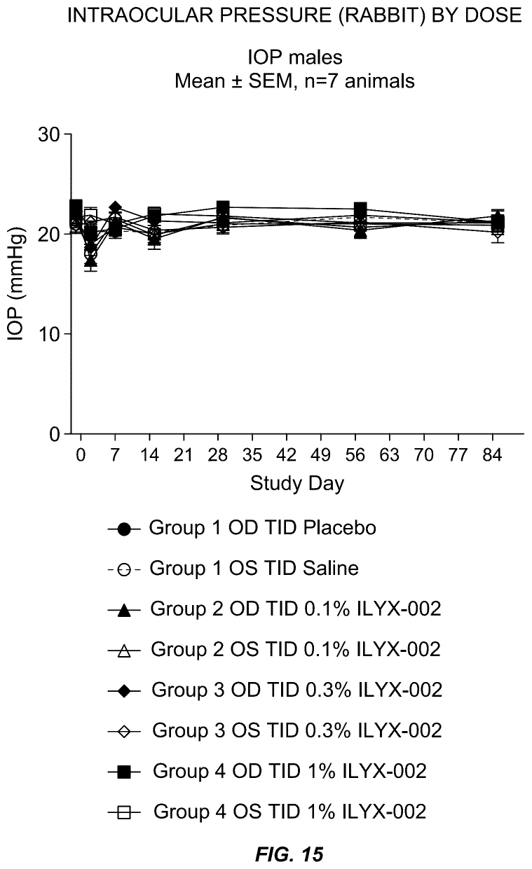

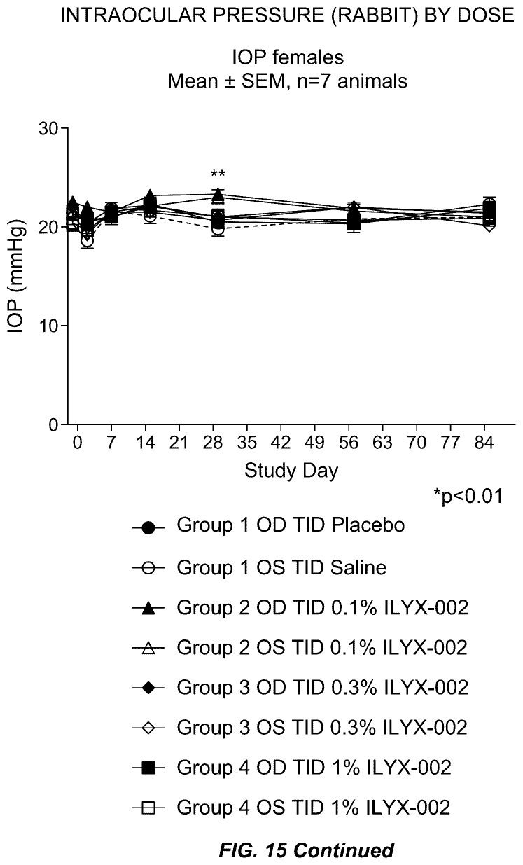

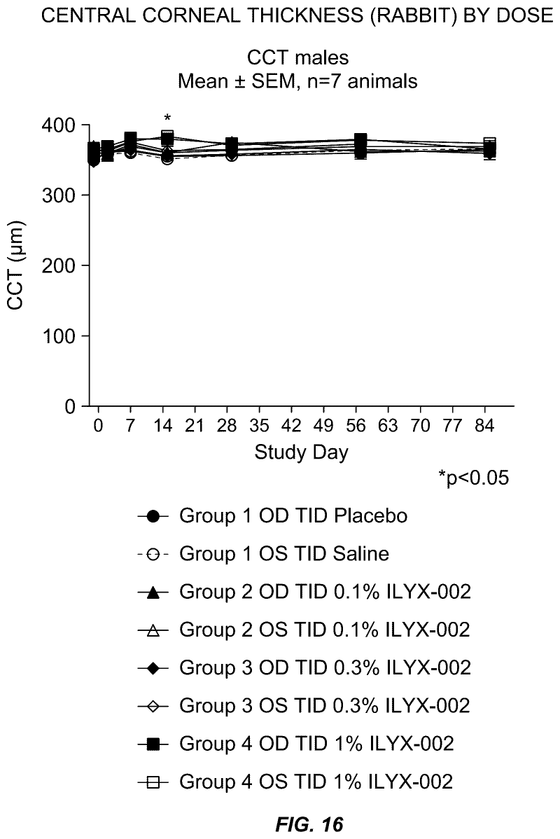

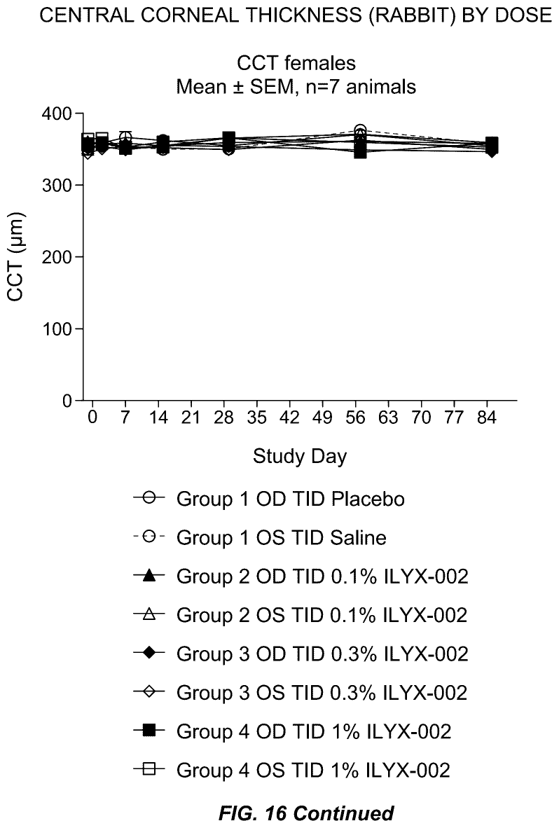

FIGS. 14, 15, and 16 provide the results of the study. FIG. 14 provides data for the clinical response of body weight. FIG. 15 provides data for the clinical response of intraocular pressure. FIG. 16 provides data for the clinical response of central corneal thickness. As illustrated in FIGS. 14, 15, and 16, the formulation of roflumilast was well tolerated at all concentrations. FIG. 14 illustrates that for all concentrations of roflumilast the rabbits did not lose weight over the course of the study as compared to vehicle-based placebo. FIG. 15 illustrates that the intraocular pressure was higher for the group administered the 0.1% w/v roflumilast formulation at day 28, however only transiently, and still within a normal range. For the remaining days, the intraocular pressure was substantially the same for all treatment groups over the duration of the study. Ophthalmic and other exams also showed that the drug was well tolerated and had little to no systemic adverse effect on the eye or other organs of the body. FIG. 16 illustrates that the central corneal thickness was substantially the same for all treatment groups. Ophthalmic and other exams also showed that the drug was well tolerated and had little to no systemic adverse effect on the eye or other organs of the body. Thus, given the wide range of concentrations tested, including those yielding higher and longer duration tissue concentrations in the anterior and vitreous chambers, it is expected that all concentrations of roflumilast would be well tolerated and produce reduced side effects relative to other standard of care treatments currently available.

The foregoing description has been presented for purposes of illustration and description. This description is not intended to limit the invention to the precise form disclosed. Persons of ordinary skill in the art will appreciate that modifications and substitutions of the basic inventive description may be made.

What is claimed is:

1. A method for treating a patient having an anterior ophthalmic disorder, comprising:
topically administering an ophthalmic pharmaceutical composition comprising 0.3% to 1.0% w/v of roflumilast or a pharmaceutically acceptable salt thereof to an ocular surface of said patient three times per day.

2. The method of claim 1, wherein the anterior ophthalmic disorder is selected from the group consisting of anterior uveitis, pan-uveitis, intermediate uveitis, uveitis associated with autoimmune or systemic diseases including juvenile idiopathic arthritis (JIA), Behçet's disease, ankylosing spondylitis, sarcoidosis, Vogt-Koyanagi-Harada disease (VKH), lupus, rheumatoid arthritis (RA), multiple sclerosis, an autoimmune condition, idiopathic uveitis, HLA-B27 uveitis, uveitic glaucoma, iritis, iridocyclitis, rubeosis iritis, coloboma of the iris, Fuch's heterchromic iridocyclitis, iridocorneal endothelial syndrome (ICE), scleritis, corneal endothelial disease or dysfunction including endotheliitis, Fuch's corneal or endothelial dystrophy, endothelial corneal dysfunction associated with Graves ophthalmopathy or thyroid eye disease megalocornea, Axenfeld's syndrome, Rieger's anomaly, Peters' anomaly, Anterior segment mesenchymal dysgenesis, ocular hypertension, glaucoma, bupthalmos, Posner Schlossman Syndrome, glaucomatocyclitic crisis, cataract, anterior post-surgical ocular inflammation, post-surgical ocular inflammation affecting corneal endothelium, deep corneal epithelial defects that also involve anterior segment dysfunction such as corneal endothelial dysfunction or neurotrophic keratitis, inflammation related to DSEK/DMET keratoplasty, rebound iritis, pseudoexfoliation syndrome, pupillary abnormalities, Stevens Johnson syndrome, Sjogren's syndrome, ocular graft-versus-host disease, ocular cicatricial pemphigoid, anterior sterile or infectious endophthalmitis, toxic anterior segment syndrome (TASS), sympathetic ophthalmia, hyphema, anterior segment ischemia, anterior chamber inflammation related to an underlying viral and other microbial cause, anterior chemical injury, and iatrogenic anterior chamber inflammation.

3. The method of claim 2, wherein the anterior ophthalmic disorder is selected from the group consisting of anterior, pan, intermediate, or posterior uveitis, uveitis associated with HLA-B27 or an autoimmune or systemic disease uveitic glaucoma, anterior chamber iatrogenic inflammation, and inflammatory disease of the iris, sclera, or choroid.

4. The method of claim 1, wherein said administration results in a reduction of at least one side effect relative to administration of a corticosteroid.

5. The method of claim 1, wherein said administration results in a reduction of dose frequency relative to the administration of a corticosteroid.

6. The method of claim 1, wherein said administration results in a significant increase in anterior tissue concentration of roflumilast at a therapeutic level.

7. The method of claim 1, wherein the ophthalmic pharmaceutical composition is a suspension.

8. A method for treating a patient having an anterior ophthalmic disorder, comprising:
topically administering an ophthalmic pharmaceutical composition comprising 0.3% to 1.0% w/v of roflumilast or a pharmaceutically acceptable salt thereof to an ocular surface of said patient four times per day.

9. The method of claim 8, wherein the anterior ophthalmic disorder is selected from the group consisting of anterior compartment disorders including anterior uveitis, pan-uveitis, intermediate uveitis, uveitis associated with autoimmune or systemic diseases including juvenile idiopathic arthritis (JIA), Behçet's disease, ankylosing spondylitis, sarcoidosis, Vogt-Koyanagi-Harada disease (VKH), lupus, rheumatoid arthritis (RA), multiple sclerosis, an autoimmune condition, idiopathic uveitis, HLA-B27 uveitis, uveitic glaucoma, iritis, iridocyclitis, rubeosis iritis, coloboma of the iris, Fuchs heterchromic iridocyclitis, iridocorneal endothelial syndrome (ICE), scleritis, corneal endothelial disease or dysfunction including endotheliitis, Fuch's corneal or endothelial dystrophy, endothelial corneal dysfunction associated with Graves ophthalmopathy or thyroid eye disease, megalocornea, Axenfeld's syndrome, Rieger's anomaly, Peters' anomaly, anterior segment mesenchymal dysgenesis, ocular hypertension, glaucoma, bupthalmos, Posner Schlossman Syndrome, glaucomatocyclitic crisis, cataract, anterior post-surgical ocular inflammation, post-surgical ocular inflammation affecting corneal endothelium, deep corneal epithelial defects that also involve anterior segment dysfunction such as corneal endothelial dysfunction or neurotrophic keratitis, inflammation related to DSEK/DMET keratoplasty, rebound iritis, pseudoexfoliation syndrome, pupillary abnormalities, Stevens Johnson syndrome, Sjogren's syndrome, ocular graft-versus-host disease, ocular cicatricial pemphigoid, anterior sterile or infectious endophthalmitis, toxic anterior segment syndrome (TASS), sympathetic ophthalmia, hyphema, anterior segment ischemia, anterior chamber inflammation related to the underlying viral and other microbial causes, anterior chemical injury, and iatrogenic anterior chamber inflammation.

10. The method of claim 8, wherein said administration results in a reduction of at least one side effect relative to administration of a corticosteroid.

11. The method of claim 8, wherein said administration results in a reduction of dosing frequency relative to administration of a corticosteroid.

12. The method of claim 8, wherein said administration results in a significant increase in anterior tissue concentration of roflumilast at a therapeutic level.

13. The method of claim 12, wherein the ophthalmic pharmaceutical composition is a suspension.

14. A method for treating a patient having a vitreous ophthalmic disorder, comprising:
topically administering an ophthalmic pharmaceutical composition comprising 0.3% to 1.0% w/v of roflumilast or a pharmaceutically acceptable salt thereof to an ocular surface of said patient three or four times per day.

15. The method of claim 14, wherein the vitreous ophthalmic disorder is selected from the group consisting of posterior, pan or intermediate uveitis, uveitis associated with HLA-B27, juvenile idiopathic arthritis, Behcets disease, ankylosing spondylitis, VKH, or an other autoimmune disease, diabetic retinopathy, diabetic macular edema, retinal vein occlusion, age-related macular degeneration including dry, geographic atrophy, exudative AMD, choroidal neovascularization, choroidal thickening associated with thyroid eye disease, Coat's disease, central serous retinopathy or chorioretinopathy, sterile or infectious endopthalmitis, inflammation associated with inherited retinal diseases, retinitis pigmentosa, Stargardt disease, Leber congenital amaurosis, Leber hereditary optic neuropathy, Usher syndrome, X-linked retinoschisis, choroidemia, zonal occult outer retinopathy, myopia, vitreomacular adhesion, retinal detachment, choroidal detachment and hemorrhage, choroidal rupture, choroidal folds, proliferative vitreoretinopathy, idiopathic ischemia, achromatopsia, retinopathy of prematurity, gyrate atrophy, central areolar choroidal dystrophy, punctate inner choroidopathy, multifocal choroiditis, choroiditis, choroidal granuloma, choroidal dystrophy, choroidal fibrosis, acute posterior multifocal placoid pigment epitheliopathy, serpiginous choroidopathy, birdshot retinochoroidopathy, multiple evanescent white dot syndrome, retinoblastoma, choroidal melanoma, retinal lymphoma, and iatrogenic posterior or vitreous chamber inflammation.

16. The method of claim 15, wherein the vitreous ophthalmic disorder is selected from the group consisting of posterior, pan or intermediate uveitis, uveitis associated with HLA-B27, juvenile idiopathic arthritis, Behcets disease, ankylosing spondylitis, VKH, or an other autoimmune disease, diabetic retinopathy, diabetic macular edema, age-related macular degeneration including dry, geographic atrophy, exudative AMD, choroidal neovascularization, choroidal thickening associated with thyroid eye disease, sterile or infectious endophthalmitis, and vitreous or posterior chamber iatrogenic inflammation.

17. The method of claim 14, wherein said administration results in a reduction of at least one side effect relative to administration of a corticosteroid.

18. The method of claim 14, wherein said administration results in a reduction of dosing frequency relative to administration of a corticosteroid.

19. The method of claim 14, wherein said administration results in a significant increase in vitreous concentration of roflumilast at a therapeutic level.

20. The method of claim 14, wherein said topical administration is three times per day.

21. The method of claim 14, wherein said topical administration is four times per day.
